SEQUENCE LISTING (6 sequences)

SEQ ID NO: 1 - DNA, 1536 bp, Vesicular Stomatitis Indiana Virus
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 2 - DNA, 1554 bp, Vesicular Stomatitis New Jersey Virus
atgttgtcttatctaatctttgcacttgccgtttcgcccattttgggcaaaattgaaattgtgtttcctcaacataccactggggattggaagagagttccccatgaatataattattgccctaccagcgcagacaagaactcacatgggactcaaacaggaatccctgttgagttaacaatgccaaaaggactaacaacccatcaagttgaaggatttatgtgtcactcagccttgtggatgaccacttgtgacttcagatggtatgggccaaaatacataacccattccatacataatgaagagcctacagattatcaatgtttggaggccattaagtcatacaaagatggagtcagtttcaatccagggtttcctcctcagagctgcgggtatggcacagttaccgatgccgaagcccatattgtgacagttactccccactctgtcaaagtggacgagtacacgggggaatggatcgatccacatttcatcggaggaaggtgcaaaggacaaatttgtgaaacagtccataattccacaaaatggtttacgtcctctgatggagaaagtgtctgcagtcaattgtttactttggttggaggaatttttttctctgattcagaagagattacctccatggggttaccagaaacaggaatcagaagtaattacttcccctacatatctacagagggaatttgcaaaatgccgttttgcagaaaacaggggtacaagcttaaaaatgacctctggttccagatcatggacccagacctggataaaacggttagagatctccctcatattaaggactgtgacctctcctcgtccataatcacaccaggagaacatgctacagacatctcactgatatcagatgttgaaaggatcctggactatgctctttgtcagaatacatggagtaaaattgaatcgggagaaccaattactccggtagatctcagctatcttgggccaaaaaacccaggggttgggccggtcttcaccatcattaacggttccctgcattattttacatcgaagtatctgcgagtcgaattagaaagtcctgtcatacccagaatggaaggaaaagttgcaggaactaggattgtacggcaattgtgggatcagtggtttcctttcggagaagttgagattggacccaatggtgtgttgaaaacgaagcaagggtataaattcccactacacatcattggaactggagaagtagacagtgacatcaaaatggaaagggttgtcaagcactgggaacacccccatattgaggccgctcagacatttttaaaaaaagatgacacaggagaagtcctttattatggcgacaccggagtgtcgaaaaatccagttgaattagtcgagggatggtttagtggatggaggagctccctcatgggagtgctggctgtgattataggatttgtgattttaatgtttttaattaaattgattggagtcttatctagccttttcagacctaaacgcaggccaatctacaaatcagacgtggaaatggctcatttccgttaa

SEQ ID NO: 3 - DNA, 1536 bp, Artificial
  Codon Optimized VSV G protein coding sequence, Indiana serotype
atgaagtgcctgctgtacctggccttcctgttcatcggcgtgaactgcaagttcaccatcgtgttcccccacaaccagaagggcaactggaagaacgtgcccagcaactaccactactgccccagcagcagcgacctgaactggcacaacgacctgatcggcaccgccctgcaagtgaagatgcccaagagccacaaggccatccaggccgacggctggatgtgccacgccagcaagtgggtgaccacctgcgacttccggtggtacggccccaagtacatcacccacagcatccgcagcttcacccccagcgtggagcagtgcaaggagagcatcgagcagaccaagcagggcacctggctgaaccccggcttccccccccaaagctgcggctacgccaccgtgaccgacgccgaggccgtgatcgtgcaggtgaccccccaccacgtgctggtggacgagtacaccggcgagtgggtggacagccagttcatcaacggcaagtgcagcaactacatctgccccaccgtgcacaacagcaccacctggcacagcgactacaaggtgaagggcctgtgcgacagcaacctgatcagcatggacatcacgttcttcagcgaggacggcgagctgagcagcctgggcaaggagggcaccggcttccgcagcaactacttcgcctacgagaccggcggcaaggcctgcaagatgcagtactgcaagcactggggcgtgcgcctgcccagcggcgtgtggttcgagatggccgacaaggacctgttcgccgccgcccgcttccccgagtgccccgagggcagcagcatcagcgcccccagccagaccagcgtggacgtgagcctgatccaggacgtggagcgcatcctggactacagcctgtgccaggagacctggagcaagatccgcgccggcctgcccatcagccccgtggacctgagctacctggcccccaagaaccccggcaccggccccgccttcaccatcatcaacggcaccctgaagtacttcgagacccgctacatccgcgtggacatcgccgcccccatcctgagccgcatggtgggcatgatcagcggcaccaccaccgagcgcgagctgtgggacgactgggccccctacgaggacgtggagatcggccccaacggcgtgctgcgcaccagcagcggctacaagttccccctgtacatgatcggccacggcatgctggacagcgacctgcacctgagcagcaaggcccaggtgttcgagcacccccacatccaggacgccgccagccagctgcccgacgacgagagcctgttcttcggcgacaccggcctgagcaagaaccccatcgagctggtggagggctggttcagcagctggaagagcagcatcgccagcttcttcttcatcatcggcctgatcatcggcctgttcctggtgctgcgcgtgggcatccacctgtgcatcaagctgaagcacaccaagaagcgccagatctacaccgacatcgagatgaaccgcctgggcaagtaa

SEQ ID NO: 4 - DNA, 1539 bp, Artificial
  RNA Optimized VSV G protein coding sequence, Indiana serotype
atgaagtgcctcctgtacctcgccttcctgttcatcggcgtcaactgcaagttcacgatcgtcttcccgcacaaccagaagggcaactggaagaacgtgccctcgaactaccactactgcccgtcgtcgagcgacctgaactggcacaacgacctgatcggcacggcgctccaagtcaagatgcccaagagccacaaggcgatccaggcggacggctggatgtgccacgcgtccaaatgggtcaccacctgcgacttccgttggtatggaccgaagtacatcacgcactccatccggtccttcactccctccgtggagcagtgcaaggagagcatcgagcagacgaagcagggcacgtggctgaaccccgggttcccgccccaaagctgcggctacgcgactgtgacggacgccgaggcggtgatcgtccaagtgacgccgcaccacgtgctggtggacgagtacacgggcgagtgggtggactcgcagttcatcaacggcaagtgctccaactacatctgccccacggtccacaactcgacgacctggcactcggactacaaggtcaaggggttgtgcgacagcaacctcatctccatggacatcaccttcttctcggaggacggcgagctctcgtccctggggaaggagggcacggggttccggagcaactacttcgcgtacgagaccggcgggaaggcctgcaagatgcagtactgcaagcactggggcgtccggctcccctcgggtgtctggttcgagatggcggacaaggacctcttcgcggcagcccggttcccggagtgcccggaggggtcgtccatcagcgctccgtcgcaaacctcggtggacgtatcgctcatccaggacgtcgagaggatcctggactactcgttgtgccaagagacctggagcaagatcagggcggggctgccgatctcgccggtggacctcagctacctcgcgccgaagaacccaggcaccggtcctgccttcaccatcatcaatggcaccctcaagtacttcgagacccgctacatccgggtggacatcgccgcgccgatcctgtcgagaatggtcggcatgatcagcgggacgaccacggagcgggagctgtgggacgactgggcgccctacgaggacgtggagatcggacccaacggcgtcctgaggaccagctccggctacaagttccccttgtacatgatcggccacggcatgctggactccgacctccacctcagctcgaaggcccaggtgttcgagcacccgcacatccaagacgctgcgtcgcagctgccggacgacgagtcgctgttcttcggcgacaccgggctatccaagaacccgatcgagctcgtggagggctggttcagttcgtggaagagctcgatcgcctcgttcttcttcatcatcgggctgatcatcggcctgttcttggtgctccgcgtcggcatccacctgtgcatcaagctgaagcacaccaagaagaggcagatctacacggacatcgagatgaaccggctcgggaagtgataa

SEQ ID NO: 5 - DNA, 1557 bp, Artificial
  RNA Optimized VSV G protein coding sequence, New Jersey serotype
atgctctcgtacctcatcttcgcgctcgccgtctcgcccatcctgggcaagatcgagatcgtgttcccgcagcacaccacgggggactggaagcgggttccccacgagtacaactactgcccgaccagcgcggacaagaactcccacgggactcagacagggatcccggtcgagctgacgatgccgaaggggctgacgacccaccaggttgagggcttcatgtgccactcggccttgtggatgaccacgtgcgacttccggtggtacgggccgaagtacatcacccactccatccacaacgaggagcccacggactaccagtgcctggaggccatcaagtcctacaaggacggagtcagcttcaacccggggttcccgccccagtcctgcggctacggcaccgtcaccgacgcggaggcccacatcgtgacggtcacgccccactccgtcaaggtggacgagtacacgggggagtggatcgacccgcacttcatcggcgggcgctgcaagggccagatctgtgagacggtccacaactccaccaagtggttcacgtcctcggacggcgagagcgtctgcagccagctgttcaccctcgtcggaggcatcttcttctcggactcggaggagatcacctccatggggctcccggagacggggatccggagcaactacttcccctacatctccaccgaggggatctgcaagatgccgttctgccgcaagcagggctacaagctcaagaacgacctctggttccagatcatggacccggacctggacaagacggttcgggacctcccgcacatcaaggactgcgacctctccagctccatcatcaccccgggcgagcacgcgacggacatctcgctgatctcagacgtcgagcggatcctggactacgcgctctgccagaacacgtggtccaagatcgagtcgggcgagccgatcacgccggtagacctcagctacctcgggccgaagaacccgggggttgggccggtcttcaccatcatcaacggctccctgcactacttcacgtcgaagtacctgcgggtcgagctggagagcccggtcatccccaggatggaggggaaggttgcgggcactcggatcgtacggcagctgtgggaccagtggttccccttcggggaggtcgagatcggacccaacggcgtgctcaagacgaagcaggggtacaagttcccgctacacatcatcggcacgggcgaggtagacagcgacatcaagatggagcgggttgtcaagcactgggagcacccccacatcgaggccgcgcagaccttcctcaagaaggacgacacaggcgaggtcctctactacggcgacaccggcgtgtcgaagaaccccgtcgagctcgtcgagggctggttcagcggctggcggagctccctcatgggcgtgctggcggtgatcatcgggttcgtgatcctgatgttcctcatcaagctgatcggcgtcctgtcgagcctcttccggcccaagcgcaggccgatctacaagtcggacgtggagatggcgcacttccggtgataa

SEQ ID NO: 6 - protein, 29 aa, Artificial
  Cytoplasmic domain of wild-type VSV G protein
RVGIHLCIKLKHTKKRQIYTDIEMNRLGK

(19) United States
(12) Patent Application Publication
(10) Pub. No.: US 2009/0175900 A1
(43) Pub. Date: Jul. 9, 2009

(54) METHODS FOR PACKAGING PROPAGATION-DEFECTIVE VESICULAR STOMATITIS VIRUS VECTORS

(75) Inventors: Christopher L. Parks, Boonton, NJ (US); Susan E. Witko, New City, NY (US); Maninder K. Sidhu, New City, NY (US); J. Erik Johnson, Verona, NJ (US); Roger Michael Hendry, Atlanta, GA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/335,763

(22) Filed: Dec. 16, 2008

Related U.S. Application Data
(60) Provisional application No. 61/015,375, filed on Dec. 20, 2007.

(51) Int. Cl. A61K 39/12; C12N 15/09; C12N 15/87; C12N 15/63

(52) U.S. Cl. 424/199.1; 435/455; 435/461; 435/320.1

(57) ABSTRACT

A method of producing propagation-defective Vesicular Stomatitis Virus (VSV) in a cell culture is provided. The method involves introducing a plasmid vector encoding an optimized VSV G gene into a cell; expressing VSV G protein from the optimized VSV G gene; and introducing a propagation-defective VSV into the cell expressing the VSV G protein encoded by the optimized VSV G gene. The method further includes growing the cells in culture; and recovering the propagation-defective VSV from the culture.

### CROSS-REFERENCE TO RELATED APPLICATIONS

This application claims the benefit of U.S. provisional application No. 61/015,375, filed Dec. 20, 2007, the entire contents of which are incorporated herein by reference.

### FIELD OF THE INVENTION

The present invention relates generally to negative-strand RNA viruses. In particular, the invention relates to methods and compositions for producing attenuated Vesicular stomatitis virus (VSV) in a cell culture.

### BACKGROUND TO THE INVENTION

Vesicular stomatitis virus (VSV) is a member of the Rhabdoviridae family, and as such is an enveloped virus that contains a non-segmented, negative-strand RNA genome. Its relatively simple genome consists of 5 gene regions arranged sequentially 3′-N-P-M-G-L-5′ (FIG. 1) (Rose and Whitt, Rhabdoviridae: The Viruses and Their Replication. In “Fields Virology”, 4^{th} Edition, Vol. 1. Lippincott and Williams and Wilkins, 1221-1244, 2001).

The N gene encodes the nucleocapsid protein responsible for encapsidating the genome while the P (phosphoprotein) and L (large) coding sequences specify subunits of the RNA-dependent RNA polymerase. The matrix protein (M) promotes virion maturation and lines the inner surface of the virus particle. VSV encodes a single envelope glycoprotein (G), which serves as the cell attachment protein, mediates membrane fusion, and is the target of neutralizing antibodies.

VSV has been subjected to increasingly intensive research and development efforts because numerous properties make it an attractive candidate as a vector in immunogenic compositions for human use (Bukreyev, et al. J. Virol. 80:10293-306, 2006; Clarke, et al. Springer Semin Immunopathol. 28: 239-253, 2006). These properties include: 1) VSV is not a human pathogen; 2) there is little pre-existing immunity that might impede its use in humans; 3) VSV readily infects many cell types; 4) it propagates efficiently in cell lines suitable for manufacturing immunogenic compositions; 5) it is genetically stable; 6) methods exist by which recombinant virus can be produced; 7) VSV can accept one or more foreign gene inserts and direct high levels of expression upon infection; and 8) VSV infection is an efficient inducer of both cellular and humoral immunity. Once reverse-genetics methods (Lawson, et al. Proc Natl Acad Sci USA 92:4477-81, 1995; Schnell, et al. EMBO J 13:4195-203, 1994) were developed, that made it possible to engineer recombinant VSV (rVSV), the first vectors were designed with foreign coding sequence inserted between the G and L genes (FIG. 1) along with the requisite intergenic transcriptional control elements. These prototype vectors were found to elicit potent immune responses against the foreign antigen and were well tolerated in the animal models in which they were tested (Grigera, et al. Virus Res 69:3-15, 2000; Kahn et al. J Virol 75:11079-87, 2001; Roberts, et al. J Virol 73:3723-32, 1999; Roberts, et al. J Virol 72:4704-11, 1998, Rose, et al. Cell 106:539-49, 2001; Rose, et al. J Virol 74:10903-10, 2000; Schlereth, et al. J Virol 74:4652-7, 2000). Notably, Rose et al. found that coadministration of two vectors, one encoding HIV-1 env and the other encoding SIV gag, produced immune responses in immunized macaques that protected against challenge with a pathogenic SHIV (Rose, et al. Cell 106:539-49, 2001).

Encouraging preclinical performance by prototype viruses has led to the development of rVSV vectors for use in humans (Clarke, et al. Springer Semin Immunopathol 28:239-253, 2006). Investigation of highly attenuated vectors is receiving considerable attention because they should offer enhanced safety profiles. This is particularly relevant since many immunogenic compositions under consideration might be used in patients with compromised immune systems (i.e. HIV-infected subjects).

The desire to develop highly attenuated vectors has focused some attention on propagation-defective rVSV vectors. Ideally, propagation-defective vectors are engineered with genetic defects that block virus propagation and spread after infection, but minimally disturb the gene expression apparatus allowing for adequate antigen synthesis to induce protective immune responses. With this objective in mind, propagation-defective rVSV vectors have been produced through manipulation of the VSV G, which is the viral attachment protein (G; FIG. 2). Vectors have been developed encoding a variety of antigens and molecular adjuvants in which the G gene has been deleted completely (VSV-ΔG) or truncated to encode a G protein lacking most of the extracellular domain (VSV-Gstem) (Clarke, et al. Springer Semin Immunopathol 28:239-253, 2006), Kahn et al. J Virol 75:11079-87, 2001; Klas, et al. Vaccine 24:1451-61, 2006; Klas, et al. Cell Immunol 218:59-73, 2002; Majid, et al. J Virol 80:6993-7008, 2006; Publicover, et al. J Virol 79:13231-8, 2005) (Wyeth unpublished data). Propagation-defective vectors such as VSV-Gstem and VSV-ΔG, do not encode functional attachment proteins, and must be packaged in cells that express G protein.

Although the ΔG and Gstem vectors are promising, the development of scaleable propagation methods that are compliant with regulations governing manufacture of immunogenic compositions for administration to humans remains a hurdle that must be addressed before clinical evaluation can be justified. A viable production method must provide sufficient quantities of functional G protein in trans to stimulate morphogenesis or “packaging” of infectious virus particles. Achieving satisfactory levels of G protein expression is complicated by the fact that G is toxic to cell lines, in part because it mediates membrane fusion (Rose and Whitt, Rhabdoviridae: The Viruses and Their Replication. In “Fields Virology”, 4^{th} Edition, Vol. 1. Lippincott Williams and Wilkins, 1221-1244, 2001). This toxicity prevents development of complementing cell lines that constitutively express the viral glycoprotein. Similarly, development of stable cell lines that express G protein from an inducible promoter is problematic because leaky expression frequently results in toxicity, and levels achieved after induction often are insufficient to promote efficient packaging particularly on a scale needed for manufacturing immunogenic compositions. One inducible cell line has been described (Schnell, et al. Cell 90:849-57, 1997), but it often loses its ability to express G protein after several passages and is derived from BHK cells, which are not a cell type presently qualified for production of immunogenic compositions for human administration. Transient production of G protein in transfected BHK (Majid, et al. J Virol 80:6993-7008, 2006) or 293T (Takada, et al. Proc Natl Acad Sci USA 94:14764-9, 1997) cells or electroporated Vero cells (Witko, et al. J Virol Methods 135:91-101, 2006) has been used to propagate propagation-defective VSV, as well.

Prior to the present invention, transient G protein expression was proven adequate to produce relatively small-scale quantities of rVSV-ΔG and rVSV-Gstem vectors needed for preclinical studies. However, these prior methods presently are inadequate for clinical development because the published procedures routinely rely on cell lines that are not qualified for production for use in humans (i.e. BHK) or the protocols have not been adapted and optimized for large-scale manufacture. Furthermore, observed yields of viral particles with these prior methods generally are less than 1×10⁷ IUs per ml (data not shown), and given that a single human dose is expected to be at least 1×10⁷ IUs per ml, manufacturing of a VSV vector will be practical only if greater than 10⁷ IUs are produced per ml of culture medium.

Therefore, there is a need in the art for methods of producing attenuated VSV particles, wherein the yields of attenuated VSV particles recovered are sufficient to be of use in manufacture of immunogenic compositions. Also, such methods would employ cells qualified for production for administration to humans.

### SUMMARY OF THE INVENTION

The present invention provides a method of producing attenuated Vesicular Stomatitis Virus (VSV) in a cell culture. The method comprises introducing a plasmid vector comprising an optimized VSV G gene into cells; expressing VSV G protein from said optimized VSV G gene; infecting the cells expressing VSV G protein with an attenuated VSV; growing the infected cells in culture; and recovering the attenuated VSV from the culture. In some embodiments of this method, the attenuated VSV is a propagation-defective VSV.

In one embodiment of the method described above, the infecting step comprises coculturing the cells expressing the VSV G protein from the optimized VSV G gene with cells transfected with: a viral cDNA expression vector comprising a polynucleotide encoding a genome or antigenome of the attenuated VSV; one or more support plasmids encoding an N, P, L and G protein of VSV; and a plasmid encoding a DNA dependent RNA polymerase. In certain embodiments of this method, the cells are further transfected with a support plasmid encoding an M protein of VSV. In some preferred embodiments, the cells are transfected via electroporation.

The present invention provides a further method of producing attenuated Vesicular Stomatitis Virus (VSV) in a cell culture. The method includes: transfecting cells (e.g., by electroporation) with: a viral cDNA expression vector comprising a polynucleotide encoding a genome or antigenome of the attenuated VSV; one or more support plasmids encoding N, P, L and G proteins of VSV; and a plasmid encoding a DNA dependent RNA polymerase; growing the transfected cells in culture; rescuing the attenuated VSV from the culture; infecting cells expressing VSV G protein encoded by an optimized VSV G gene with the rescued attenuated VSV; growing the infected cells in culture; and recovering the attenuated VSV from the culture of infected cells. In certain embodiments, the viral rescue cells are further transfected with a support plasmid encoding a VSV M protein. In some embodiments of this method, the attenuated VSV is a propagation-defective VSV.

The invention also provides a method of improving the packaging of a propagation-defective Vesicular Stomatitis Virus (VSV). This method includes introducing (e.g. by transfection) a plasmid vector encoding an optimized VSV G gene into a cell; transiently expressing VSV G protein from the optimized VSV G gene; introducing a propagation-defective VSV into the cell transiently expressing the VSV G protein; growing cells in culture; and recovering the packaged VSV from the culture. The propagation-defective VSV may be introduced into the cells transiently expressing the VSV G protein by, for example, infecting such cells with the propagation-defective VSV. In some embodiments, this infection is achieved by coculturing the cells expressing the VSV G protein with cells transfected (e.g., by electroporation) with: a viral cDNA expression vector comprising a polynucleotide encoding a genome or antigenome of the propagation-defective VSV; one or more support plasmids encoding an N, P, L and G protein of VSV; and a plasmid encoding a DNA dependent RNA polymerase. In certain embodiments, the cells are further transfected with a support plasmid encoding an M protein of VSV.

In preferred embodiments, the methods of the present invention employ cells that are qualified production cells. In some embodiments, the qualified production cells are Vero cells.

In some embodiments of the methods of the present invention, viral genome-length RNA is transcribed from the polynucleotide encoding the genome or antigenome of the attenuated VSV. In some embodiments, the polynucleotide is operatively linked to a transcription terminator sequence. In some further embodiments, the polynucleotide is operatively linked to a ribozyme sequence.

In some preferred embodiments of the methods of the present invention, the DNA-dependent RNA polymerase is T7 RNA polymerase and the viral cDNA expression vector and the support plasmids are under the control of a T7 promoter.

In certain embodiments of the methods of this invention, the VSV G protein encoded by the support plasmid is encoded by a non-optimized VSV G gene. In other embodiments, the VSV G protein encoded by the support plasmid is encoded by an optimized VSV G gene.

In some embodiments of the methods of the invention, the expression of VSV G protein from the optimized VSV G gene is under the control of a cytomegalovirus-derived RNA polymerase II promoter. In some further embodiments of the instant methods, the expression of VSV G protein from the optimized VSV G gene is under the control of a transcriptional unit recognized by RNA polymerase II producing a functional mRNA.

In certain embodiments, the optimized VSV G gene employed in the methods of the present invention is derived from an Indiana VSV serotype or New Jersey VSV serotype. In some embodiments, the optimized VSV G gene employed in the methods of the invention is selected from the following: SEQ ID NO: 3, SEQ ID NO: 4 and SEQ ID NO: 5.

In some embodiments, the attenuated VSV produced by the methods of this invention encodes a heterologous antigen. The heterologous antigen may be from a pathogen, for example. In some embodiments, the pathogen may be selected from, but is not limited to, the following: measles virus, subgroup A and subgroup B respiratory syncytial viruses, human parainfluenza viruses, mumps virus, human papilloma viruses of type 1 or type 2, human immunodeficiency viruses, herpes simplex viruses, cytomegalovirus, rabies virus, human metapneumovirus, Epstein Barr virus, filoviruses, bunyaviruses, flaviviruses, alphaviruses, influenza viruses, hepatitis C virus and C. trachomatis.

In some embodiments of the instant methods, the attenuated VSV further encodes a non-viral molecule selected from a cytokine, a T-helper epitope, a restriction site marker, or a protein of a microbial pathogen or parasite capable of eliciting an immune response in a mammalian host.

In one embodiment of the methods of the present invention, the attenuated VSV lacks a VSV G protein (VSV-ΔG). In certain embodiments, the yield of VSV-ΔG using the methods of the present invention is greater than about 1×10⁶ IU per ml of culture.

In some other embodiments of the methods of this invention, the attenuated VSV expresses a G protein having a truncated extracellular domain (VSV-Gstem). In certain embodiments, the yield of VSV-Gstem using the methods of this invention is greater than about 1×10⁶ IU per ml of culture.

In some further embodiments of the instant methods, the attenuated VSV expresses a G protein having a truncated cytoplasmic tail (CT) region. In certain embodiments, the attenuated VSV expresses a G protein having a cytoplasmic tail region truncated to one amino acid (G-CT1). In other particular embodiments, the attenuated VSV expresses a G protein having a cytoplasmic tail region truncated to nine amino acids (G-CT9).

In further embodiments of the instant methods, the attenuated VSV includes the VSV N gene that has been translocated downstream from its wild-type position in the viral genome, thereby resulting in a reduction in VSV N protein expression. In still further embodiments of the methods of this invention, the attenuated VSV contains noncytopathic M gene mutations (Mncp), said mutations reducing the expression of two overlapping in-frame polypeptides that are expressed from the M protein mRNA by initiation of protein synthesis at internal AUGs, affecting IFN induction, affecting nuclear transport, or combinations thereof.

The present invention further provides an immunogenic composition including an immunogenically effective amount of attenuated VSV produced according to any of the instant methods in a pharmaceutically acceptable carrier. In some embodiments of the immunogenic composition, the attenuated VSV encodes a heterologous antigen.

Also provided by the present invention is a composition for producing an attenuated Vesicular Stomatitis Virus (VSV) in a cell culture. The composition includes a vector including an optimized VSV G gene; a polynucleotide encoding a genome or antigenome of an attenuated VSV; and a vector that encodes a DNA-dependent RNA polymerase. In another embodiment, the composition further includes one or more support vectors that encode VSV proteins selected from: an N protein; a P protein; an L protein; an M protein; and a G protein.

In some embodiments of the composition, the DNA-dependent RNA polymerase encoded by the polynucleotide encoding the genome or antigenome of the attenuated VSV is a T7 RNA polymerase. In some further embodiments, said polynucleotide encodes the genome or antigenome of a propagation-defective VSV.

The present invention also provides a kit for producing an attenuated Vesicular Stomatitis Virus (VSV) in a cell culture. The kit at least includes a vector that includes an optimized VSV G gene.

The kit may further contain a viral cDNA expression vector that includes a polynucleotide encoding a genome or antigenome of an attenuated VSV; and a vector that encodes a DNA-dependent RNA polymerase. In some embodiments, the DNA-dependent RNA polymerase is T7 RNA polymerase. In certain embodiments, the kit further includes one or more support vectors that encode VSV proteins selected from: an N protein; a P protein; an L protein; an M protein; and a G protein.

### BRIEF DESCRIPTION OF THE SEQUENCES

SEQ ID NO: 1 Coding sequence for native VSV G protein (Indiana serotype);

SEQ ID NO: 2 Coding sequence for native VSV G protein (New Jersey serotype);

SEQ ID NO: 3 Codon optimized VSV G protein coding sequence (opt1; Indiana serotype);

SEQ ID NO: 4 RNA optimized VSV G protein coding sequence (RNAopt; Indiana serotype);

SEQ ID NO: 5 RNA optimized VSV G protein coding sequence (RNAopt; New Jersey serotype); and

SEQ ID NO: 6 cytoplasmic domain of wild-type VSV G protein.

### BRIEF DESCRIPTION OF THE DRAWINGS

FIG. 1 is a schematic representation of the RNA genome of Vesicular Stomatitis Virus (VSV). The VSV genome encodes Nucleocapsid (N), Phosphoprotein (P), Matrix protein (M), Glycoprotein (G) and Large Protein (L).

FIG. 2 shows schematic representations of examples of propagation-defective VSV vectors (VSV-Gstem and VSV-ΔG) suitable for use in the methods of the present invention. The HIV Gag coding sequence is used as an example of a foreign gene.

FIG. 3 shows a VSV G protein coding sequence for the Indiana serotype obtained by the RNA optimization method described herein. Lower case letters indicate substitutions made during optimization. An Xho I (5′) restriction site (i.e., ctcgag) and Xba I (3′) restriction site (i.e., tctaga) were added during the optimization. An EcoR I (5′) restriction site (i.e., gaattc) was added after optimization. The region of the RNA optimized VSV G gene (Indiana) corresponding to the translated VSV G protein is represented by SEQ ID NO: 4.

FIG. 4 shows a VSV G protein coding sequence for the New Jersey serotype obtained by the RNA optimization method described herein. Lower case letters indicate substitutions made during optimization. Xho I (5′) and Xba I (3′) restriction sites were added during the optimization. An EcoR I (5′) restriction site was added after optimization. The region of the RNA optimized VSV G gene (New Jersey) corresponding to the translated VSV G protein is represented by SEQ ID NO: 5.

FIG. 5 shows a VSV G protein coding sequence for the Indiana serotype obtained by the codon optimization method (Optimization 1) described herein. An Xho I (5′) restriction site (i.e, ctcgag) and Xba I (3′) restriction site (i.e., tctaga) were added during the optimization. The VSV G protein amino acid sequence (Indiana serotype) was reverse translated using a human codon frequency table supplied in the Seq Web sequence analysis suite (Accelrys, Inc.). The sequence context of the ATG translation initiation signal (boxed; Kozak, J Biol Chem 266:19867-70, 1991), and translation terminator (double underlined; Kochetov, et al. FEBS Lett 440: 351-5, 1998) are shown. Four codons were modified as shown in underlining to reduce similarity with splice site consensus. The modified codons were as follows: 190 CAG to CAA (acceptor site), 277 CGC to CGG (donor site), 400 CAG to CAA (acceptor site), and 625 ACC to ACG (acceptor site). The region of the codon optimized VSV G gene (Indiana) corresponding to the translated VSV G protein is represented by SEQ ID NO: 3.

FIG. 6 Panel A shows schematic representations of plasmid vectors encoding VSV G proteins (Indiana serotype) controlled by the CMV promoter and enhancer. pCMV-Gin includes the gene for the native VSV membrane glycoprotein (Gin), whereas pCMV-Gin/Opt-1 and pCMV-Gin/RNAopt include optimized VSV G genes obtained, respectively, by either the codon optimized (Opt-1) or RNA optimized (RNAopt) methods described herein. Panel B is a Western blot analysis of G protein expression with an anti-VSV polyclonal antiserum at 24 h and 72 h post electroporation of Vero cells with pCMV-Gin/Opt1 (lanes 2 and 7, respectively), with pCMV-Gin/RNAopt (lanes 3 and 8, respectively), or with pCMV-Gin (lanes 1 and 6, respectively). VSV protein expression at 24 h and 72 h of mock transfected Vero cells (negative control) is shown in lanes 4 and 9, respectively, and of VSV-infected Vero cells (positive control) is shown in lanes 5 and 10, respectively.

FIG. 7 The top of the figure shows schematic representations of plasmid vectors encoding VSV G proteins derived from the Indiana serotype (Gin) controlled by the CMV promoter and enhancer, wherein pCMV-Gin includes the gene for the native VSV membrane glycoprotein (Gin), and pCMV-Gin/Opt-1 and pCMV-Gin/RNAopt include optimized VSV G genes obtained by the codon optimized (Opt-1) and RNA optimized (RNAopt) methods, respectively, described herein. The graph at the bottom of the figure shows a comparison of the packaging yields of rVSV-Gag1-ΔG (hatched bars) or rVSV-Gag1-Gstem (solid bars) obtained from cells electroporated with G expression plasmids including the following: the coding sequence for native VSV glycoprotein Gin (1), an optimized VSV Gin gene obtained by the Opt-1 method (2) described herein or an optimized VSV G gene obtained by the RNA Opt method (3) described herein.

FIG. 8 is a Western Blot analysis showing a comparison of transient expression of native or optimized VSV G protein coding sequences derived from the New Jersey serotype (Gnj) or Indiana serotype (Gin). The analysis was performed with an anti-VSV polyclonal antiserum at 24 h and 48 h post-electroporation of Vero cells with pCMV-Gin (lanes 3 and 4, respectively), with pCMV-Gin/RNAopt (lanes 5 and 6, respectively), with pCMV-Gnj (lanes 8 and 9, respectively), and with pCMV-Gnj/RNAopt (lanes 10 and 11, respectively). VSV protein expression of Vero mock transfected cells (negative control) are shown in lanes 2 and 7, and of Vero-VSV infected cells (positive control) is shown in lane 1.

FIG. 9 Panel A of the figure shows schematic representations of plasmid vectors encoding native or optimized VSV G protein coding sequences derived from the New Jersey serotype (Gnj) or Indiana serotype (Gin). Panel B shows a comparison of packaging yields of rVSV-Gstem-gag1 obtained from cells electroporated with the G protein expression vectors shown in Panel A, which correspond to pCMV-Gin (a), pCMV-Gin/RNAopt (b), pCMV-Gnj (c), and pCMV-Gnj/RNAopt (d).

### DETAILED DESCRIPTION OF THE INVENTION

### Definitions

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude other elements.

The term “attenuated virus” and the like as used herein refers to a virus that is limited in its ability to grow or replicate in vitro or in vivo.

The term “viral vector”, and the like refers to a recombinantly produced virus or viral particle that includes a polynucleotide to be delivered into a host cell, either in vivo, ex vivo or in vitro.

The term “polynucleotide,” as used herein, means a single or double-stranded polymer of deoxyribonucleotide or ribonucleotide bases and includes DNA and corresponding RNA molecules, including HnRNA and mRNA molecules, both sense and anti-sense strands, and comprehends cDNA, genomic DNA and recombinant DNA, as well as wholly or partially synthesized polynucleotides. An HnRNA molecule contains introns and corresponds to a DNA molecule in a generally one-to-one manner. An mRNA molecule corresponds to an HnRNA and/or DNA molecule from which the introns have been excised. A polynucleotide may consist of an entire gene, or any portion thereof. Operable anti-sense polynucleotides may comprise a fragment of the corresponding polynucleotide, and the definition of “polynucleotide” therefore includes all such operable anti-sense fragments. Anti-sense polynucleotides and techniques involving anti-sense polynucleotides are well known in the art and are described, for example, in Robinson-Benion et al. “Antisense techniques,” Methods in Enzymol. 254:363-375, 1995; and Kawasaki et al. Artific. Organs 20:836-848, 1996.

As used herein, “expression” refers to a process by which polynucleotides are transcribed into mRNA and translated into peptides, polypeptides, or proteins. If the polynucleotide is derived from genomic DNA, expression may include splicing of the mRNA, if an appropriate eukaryotic host is selected.

The terms “transient expression”, “transiently expressed” and the like is intended to mean the introduction of a cloned gene into cells such that it is taken up by the cells for the purpose of expressing a protein or RNA species, wherein the expression decays with time and is not inherited. Transfection is one approach to introduce cloned DNA into cells. Transfection agents useful for introducing DNA into cells include, for example, calcium phosphate, liposomes, DEAE dextrans, and electroporation.

The terms “constitutive expression”, “constitutively expressed” and the like means constant expression of a gene product.

The term “inducible expression” means expression of a gene product from an inducible promoter. For example, an inducible promoter may respond to a chemical inducer or heat to promote expression of the gene product.

The term “promoter” as used herein refers to a regulatory region a short distance from the 5′ end of a gene that acts as the binding site for RNA polymerase.

The term “enhancer” as used herein refers to a cis-regulatory sequence that can elevate levels of transcription from an adjacent promoter.

The term “operatively linked” refers to an arrangement of elements wherein the components so described are configured so as to perform their usual function. In some instances, the term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence when the regulatory proteins and proper enzymes are present. In some instances, certain control elements need not be contiguous with the coding sequence, so long as they function to direct the expression thereof. For example, intervening untranslated, yet transcribed sequences can be present between the promoter sequence and the coding sequence and the promoter can still be considered to be “operatively linked” to the coding sequence. Thus, a coding sequence is “operatively linked” to a transcriptional and translational control sequence in a cell when RNA polymerase transcribes the coding sequence into mRNA, which is then trans-RNA spliced and translated into the protein encoded by the coding sequence. As another example, a polynucleotide may be operatively linked with transcription terminator sequences when transcription of the polynucleotide is capable of being terminated by the transcription terminator sequences. As yet another example, a polynucleotide may be operatively linked with a ribozyme sequence when transcription of the polynucleotide affects cleavage at the ribozyme sequence.

The term “antigen” refers to a compound, composition, or immunogenic substance that can stimulate the production of antibodies or a T-cell response, or both, in an animal, including compositions that are injected or absorbed into an animal. The immune response may be generated to the whole molecule, or to a portion of the molecule (e.g., an epitope or hapten). The term may be used to refer to an individual macromolecule or to a homogeneous or heterogeneous population of antigenic macromolecules. An antigen reacts with the products of specific humoral and/or cellular immunity. The term “antigen” broadly encompasses moieties including proteins, polypeptides, antigenic protein fragments, nucleic acids, oligosaccharides, polysaccharides, organic or inorganic chemicals or compositions, and the like. The term “antigen” includes all related antigenic epitopes. Epitopes of a given antigen can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are identified by determining spatial conformation of amino acids such as by, e.g., x-ray crystallography and 2-dimensional nuclear magnetic resonance. See, e.g., Epitope Mapping Protocols, supra. Furthermore, for purposes of the present invention, an “antigen” refers to a protein that includes modifications, such as deletions, additions and substitutions (generally conservative in nature, but they may be non-conservative), to the native sequence, so long as the protein maintains the ability to elicit an immunological response. These modifications may be deliberate, as through site-directed mutagenesis, or through particular synthetic procedures, or through a genetic engineering approach, or may be accidental, such as through mutations of hosts, which produce the antigens. Furthermore, the antigen can be derived or obtained from any virus, bacterium, parasite, protozoan, or fungus, and can be a whole organism. Similarly, an oligonucleotide or polynucleotide, which expresses an antigen, such as in nucleic acid immunization applications, is also included in the definition. Synthetic antigens are also included, for example, polyepitopes, flanking epitopes, and other recombinant or synthetically derived antigens (Bergmann et al. (1993) Eur. J. Immunol. 23:2777 2781; Bergmann et al. (1996) J. Immunol. 157:3242 3249; Suhrbier, A. (1997) Immunol. and Cell Biol. 75:402 408; Gardner et al. (1998) 12th World AIDS Conference, Geneva, Switzerland, Jun. 28 Jul. 3, 1998).

The term “heterologous antigen” as used herein is an antigen encoded in a nucleic acid sequence, wherein the antigen is either not from the organism, or is not encoded in its normal position or its native form.

The terms “optimized VSV G gene”, “optimized VSV G coding sequence”, and the like as used herein refers to a modified VSV G protein coding sequence, wherein the modified VSV G protein coding sequence results in expression of VSV G protein in increased amounts relative to the native G protein open reading frame.

The term “G protein complementation” as used herein refers to a method wherein a virus is complemented by complementing cell lines, helper virus, transfection or some other means to provide lost G function.

The term “growing” as used herein refers to the in vitro propagation of cells on or in media of various kinds. The maintenance and growing of cells in the laboratory involves recreating an environment that supports life and avoids damaging influences, such as microbial contamination and mechanical stress. Cells are normally grown in a growth medium within culture vessels (such as flasks or dishes for adherent cells or constantly moving bottles or flasks for cells in suspension) and maintained in cell incubators with constant temperature, humidity and gas composition. However, culture conditions can vary depending on the cell type and can be altered to induce changes in the cells. “Expansion”, and the like as used herein, is intended to mean a proliferation or division of cells.

The terms “cell”, “host cell” and the like as used herein is intended to include any individual cell or cell culture which can be or have been recipients for vectors or the incorporation of exogenous nucleic acid molecules, polynucleotides and/or proteins. It is also intended to include progeny of a single cell. However, the progeny may not necessarily be completely identical (in morphology or in genomic or total DNA complement) to the original parent cell due to natural, accidental, or deliberate mutation. The cells may be prokaryotic or eukaryotic, and include, but are not limited to, bacterial cells, yeast cells, animal cells, and mammalian cells (e.g., murine, rat, simian or human).

The term “qualified production cells” as used herein means that the cells have been qualified successfully and used to produce immunogenic compositions or gene therapy vectors for human use. Examples of such cells include, for example, Vero cells, WI-38, PERC.6, 293-ORF6, CHO, FRhL or MRC-5.

The term “cytopathic effect” or “CPE” is defined as any detectable changes in the host cell due to viral infection. Cytopathic effects may consist of cell rounding, disorientation, swelling or shrinking, death, detachment from a surface, etc.

The term “multiplicity of infection” or “MOI” is the ratio of infectious agents (e.g., virus) to infection targets (e.g., cell).

By “infectious clone” or “infectious cDNA” of a VSV, it is meant cDNA or its product, synthetic or otherwise, as well as RNA capable of being directly incorporated into infectious virions which can be transcribed into genomic or antigenomic viral RNA capable of serving as a template to produce the genome of infectious viral or subviral particles.

As described above, VSV has many characteristics, which make it an appealing vector for immunogenic compositions. For example, VSV is not considered a human pathogen. Also, VSV is able to replicate robustly in cell culture and is unable to either integrate into host cell DNA or undergo genetic recombination. Moreover, multiple serotypes of VSV exist, allowing the possibility for prime-boost immunization strategies. Furthermore, foreign genes of interest can be inserted into the VSV genome and expressed abundantly by the viral transcriptase. Moreover, pre-existing immunity to VSV in the human population is infrequent.

The present invention provides methods of producing attenuated Vesicular Stomatitis Virus (VSV) in a cell culture. The methods of the present invention provide G protein complementation to an attenuated VSV. In some embodiments, the G protein complementation provides G function to an attenuated VSV that lacks a G protein or expresses a non-functional G protein. Such vectors must be “packaged” in cells that express G protein.

The methods of the present invention are based on achieving higher levels of transient G protein expression from plasmid DNA. The methodology has been applied to the production of Gstem and ΔG rVSV vectors producing over 1×10⁶ IUs per ml.

The instant methods are scaleable for manufacturing. In some embodiments, the methods of the present invention employ Vero cells, which are a well-characterized substrate for production of immunogenic compositions and have been used to produce a licensed rotavirus vaccine (Merck, RotaTeq (Rotavirus Vaccine, Live, Oral, Pentavalent) FDA. Online, 2006 posting date; Sheets, R. (History and characterization of the Vero cell line) FDA. Online, 2000 posting date).

### Genetic Complementation Through Transient Expression

The present invention provides a packaging procedure for attenuated VSVs. The methods of the present invention have been applied to the packaging of propagation-defective recombinant VSVs, such as VSV-ΔG and VSV-Gstem. VSV-ΔG is a vector in which the G gene has been deleted completely (Roberts, et al. J Virol 73: 3723-32, 1999), whereas VSV-Gstem is a vector in which the G gene has been truncated to encode a G protein lacking most of the extracellular domain (VSV-Gstem; Robison and Whitt J Virol 74: 2239-46, 2000). In such instances, a vector packaging procedure based on transient expression of G protein as a means to compensate for lost G function will support further clinical development of VSV vector candidates, provided several criteria are met.

Among these criteria are that all materials and procedures should be compliant with regulations governing production of immunogenic compositions for human administration. Moreover, the method used to introduce a G protein expression plasmid into the cells should be efficient and scaleable to accommodate manufacturing. Furthermore, G protein expression should be sufficient to promote efficient packaging of the Gstem or ΔG vector. Also, virus particle yields should preferably routinely achieve or exceed 1×10⁶ IUs per ml. More preferably, virus particle yields should routinely achieve or exceed 1×10⁷ IUs per ml in most instances. The compositions and methods of the present invention meet these criteria.

The present invention provides a scaleable transient expression method that reproducibly yield 1×10⁷ IU per ml. With respect to clinical development of candidate VSV vectors, transient G protein expression provides two notable advantages over complementation methods that rely on stable cell lines. First, the transient expression method of the present invention is adaptable to multiple cell types. This provides flexibility when selecting cell substrates, which should be a permissive host for vector replication. Second, a validated cell type can be used directly for transient expression without extensive further qualification or testing. A stable complementing cell line likely would require extensive testing (i.e. exhaustive adventitious agent testing, karyotyping, tumorgenicity testing) after it is derived to validate it for use in production.

It has been surprisingly discovered that G protein expression from plasmids containing optimized VSV G genes significantly improved yields of both the ΔG and Gstem propagation-defective vectors. Moreover, the yields of Gstem vector generally were notably higher when compared to the equivalent ΔG vector. Taken together, an embodiment of the present invention combining Vero cell electroporation, optimized VSV G expression plasmids, and the Gstem vector boosted yields as high as 1×10⁸ IUs, providing a feasible path by which to manufacture a propagation-defective VSV vector.

As described above, the packaging method of the present invention was found to be useful for production of propagation-defective VSV Gstem and ΔG vectors. The transient G protein expression method of the invention was capable of producing over 1×10⁷ IU per ml when packaging Gstem vectors encoding HIV gag. Packaging of a VSV ΔG vector encoding HIV gag was also tested in the transient method for G protein expression and was found to be less efficient, but yields did exceed 1×10⁷ per ml in some experiments. The fact that yields of more than 1×10⁷ IU per ml were observed for both vectors with the packaging method of the present invention, and that this was achieved with Vero cells, indicates that it is possible to produce Gstem and ΔG vectors on a manufacturing-scale.

The methods of VSV G complementation according to the present invention were applied to the production of VSV Gstem and ΔG vectors, although the present invention is not limited to these embodiments. For example, the methods of the present invention can be applied to the production of other attenuated VSV particles. Examples of various recombinant VSV vectors are provided herein.

Moreover, other propagation-defective paramyxovirus or rhabdovirus vectors (i.e. Sendai virus, measles virus, mumps virus, parainfluenza virus, or vesiculoviruses) lacking their native attachment proteins may be packaged with VSV G protein on their surface using the complementation systems described herein. In fact, VSV G protein has been shown to function as an attachment protein for replication-competent recombinant measles viruses (Spielhofer, et al. J Virol 72:2150-9, 1998) indicating that it should function similarly in the context of propagation-defective morbillivirus vectors. VSV G protein also is widely used to ‘pseudotype’ retrovirus particles, thereby providing an attachment protein that can mediate infection of a broad spectrum of cell types (Cronin, et al. Curr Gene Ther 5:387-98, 2005; Yee, et al. Methods Cell Biol 43 Pt A:99-112, 1994). The methods described herein should be adaptable to retrovirus particle production, and might significantly simplify the production and improve yields of virus particles containing VSV G protein.

The complementation method of the present invention has been developed for VSV G protein expression in Vero cells, but the technology should be readily applicable to other viruses, cell types, and complementing proteins. It particularly is worth noting that the methodology described herein circumvented the toxic nature of VSV G, allowing for efficient packaging of propagation-defective VSV vectors. This suggests that this method would be adaptable to other complementation systems that require controlled expression of a toxic protein in trans.

### Methods for Recovery of Vesicular Stomatitis Virus

General procedures for recovery of non-segmented negative-stranded RNA viruses according to the invention can be summarized as follows. A cloned DNA equivalent (which is positive-strand, message sense) of the desired viral genome is placed between a suitable DNA-dependent RNA polymerase promoter (e.g., a T7, T3 or SP6 RNA polymerase promoter) and a self-cleaving ribozyme sequence (e.g., the hepatitis delta ribozyme) which is inserted into a suitable transcription vector (e.g. a propagatable bacterial plasmid). This transcription vector provides the readily manipulable DNA template from which the RNA polymerase (e.g., T7 RNA polymerase) can faithfully transcribe a single-stranded RNA copy of the viral antigenome (or genome) with the precise, or nearly precise, 5′ and 3′ termini. The orientation of the viral DNA copy of the genome and the flanking promoter and ribozyme sequences determine whether antigenome or genome RNA equivalents are transcribed.

Also required for rescue of new virus progeny according to the invention are virus-specific trans-acting support proteins needed to encapsidate the naked, single-stranded viral antigenome or genome RNA transcripts into functional nucleocapsid templates. These generally include the viral nucleocapsid (N) protein, the polymerase-associated phosphoprotein (P) and the polymerase (L) protein.

Functional nucleocapsid serves as a template for genome replication, transcription of all viral mRNAs, and accumulation of viral proteins, triggering ensuing events in the viral replication cycle including virus assembly and budding. The mature virus particles contain the viral RNA polymerase necessary for further propagation in susceptible cells.

The present invention is directed to the recovery of attenuated VSV. Certain attenuated viruses selected for rescue require the addition of support proteins, such as G and M for virus assembly and budding. For example, the attenuated VSV may be a propagation-defective VSV vector comprising a deletion of sequence encoding either all of the G protein (ΔG) or most of the G protein ectodomain (Gstem). Both ΔG and Gstem are unable to spread beyond primary infected cells in vivo. This results in a virus that can propagate only in the presence of transcomplementing G protein.

Typically, although not necessarily exclusively, rescue of non-segmented negative-stranded RNA viruses also requires an RNA polymerase to be expressed in host cells carrying the viral cDNA, to drive transcription of the cDNA-containing transcription vector and of the vectors encoding the support proteins.

Within the present invention, rescue of attenuated VSV typically involves transfecting host cells with: a viral cDNA expression vector containing a polynucleotide encoding a genome or antigenome of the attenuated VSV; one or more support plasmids encoding N, P, L and G proteins of VSV; and a plasmid encoding a DNA-dependent RNA polymerase, such as T7 RNA polymerase. The VSV G protein encoded by the support plasmid employed during viral rescue may be encoded by a native VSV G gene. However, it is also well within the contemplation of the present invention that the VSV G protein of a support plasmid used during viral rescue may be encoded by an optimized VSV G gene. In some embodiments, the cells are also transfected with a support plasmid encoding an M protein of VSV. The transfected cells are grown in culture, and attenuated VSV is rescued from the culture. The rescued material may then be co-cultured with plaque expansion cells for further viral expansion, as described in further detail below.

The host cells used for viral rescue are often impaired in their ability to support further viral expansion. Therefore, the method of producing attenuated VSV in a cell culture typically further includes infecting plaque expansion cells with the rescued, attenuated VSV. In some embodiments of the present invention, cells expressing VSV G protein encoded by an optimized VSV G gene are infected with the rescued attenuated VSV; the infected cells are grown; and the attenuated VSV is recovered from the culture of infected cells.

In some embodiments of viral rescue, the polynucleotide encoding the genome or antigenome of the attenuated VSV is introduced into the cell in the form of a viral cDNA expression vector that includes the polynucleotide operatively linked to an expression control sequence to direct synthesis of RNA transcripts from the cDNA expression vector. In some embodiments, the expression control sequence is a suitable DNA-dependent RNA polymerase promoter (e.g., a T7, T3 or SP6 RNA polymerase promoter).

In some embodiments, the support plasmids, as well as the viral cDNA expression vector used during viral rescue are under the control of a promoter of the DNA-dependent RNA polymerase. For example, in embodiments where the RNA polymerase is T7 RNA polymerase, the support plasmids and the viral cDNA expression vector would preferably be under the control of a T7 promoter.

In some other embodiments, the expression of the DNA-dependent RNA polymerase is under the control of a cytomegalovirus-derived RNA polymerase II promoter. The immediate-early human cytomegalovirus [hCMV] promoter and enhancer is described, for e.g., in U.S. Pat. No. 5,168,062, incorporated herein by reference.

In some embodiments, the method for recovering attenuated VSV from cDNA involves introducing a viral cDNA expression vector encoding a genome or antigenome of the subject virus into a host cell, and coordinately introducing: a polymerase expression vector encoding and directing expression of an RNA polymerase. Useful RNA polymerases in this context include, but are not limited to, a T7, T3, or SP6 phage polymerase. The host cells also express, before, during, or after coordinate introduction of the viral cDNA expression vector, the polymerase expression vector and the N, P, L, M and G support proteins necessary for production of mature attenuated VSV particles in the host cell.

Typically, the viral cDNA expression vector and polymerase expression vector will be coordinately transfected into the host cell with one or more additional expression vector(s) that encode(s) and direct(s) expression of the support proteins. The support proteins may be wild-type or mutant proteins of the virus being rescued, or may be selected from corresponding support protein(s) of a heterologous non-segmented negative-stranded RNA virus. In alternate embodiments, additional viral proteins may be co-expressed in the host cell, for example a polymerase elongation factor (such as M2-1 for RSV) or other viral proteins that may enable or enhance recovery or provide other desired results within the subject methods and compositions. In other embodiments, one or more of the support protein(s) may be expressed in the host cell by constitutively expressing the protein(s) in the host cell, or by co-infection of the host cell with a helper virus encoding the support protein(s).

In more detailed aspects of the invention, the viral cDNA expression vector comprises a polynucleotide encoding a genome or antigenome of VSV operably linked to an expression control sequence to direct synthesis of viral RNA transcripts from the cDNA expression vector. The viral cDNA vector is introduced into a host cell transiently expressing an RNA polymerase and the following VSV support proteins: an N protein, a P protein, an L protein, an M protein and a G protein. Each of the RNA polymerase and the N, P, L, M and G proteins may be expressed from one or more transfected expression vector(s). Often, each of the RNA polymerase and the support proteins will be expressed from separate expression vectors, commonly from transient expression plasmids. Following a sufficient time and under suitable conditions, an assembled infectious, attenuated VSV is rescued from the host cells.

To produce infectious, attenuated VSV particles from a cDNA-expressed genome or antigenome, the genome or antigenome is coexpressed with those viral proteins necessary to produce a nucleocapsid capable of RNA replication, and render progeny nucleocapsids competent for both RNA replication and transcription. Such viral proteins include the N, P and L proteins. In the instant invention, attenuated VSV vectors with lost G function also require the addition of the G viral protein. Moreover, an M protein may also be added for a productive infection. The G and M viral proteins can be supplied by coexpression. In some embodiments, the VSV G support plasmid employed during viral rescue contains a non-optimized VSV G gene. However, in other embodiments, as described below, the VSV G support plasmid employed during viral rescue contains an optimized VSV G gene.

In certain embodiments of the invention, complementing sequences encoding proteins necessary to generate a transcribing, replicating viral nucleocapsid (i.e., L, P and N), as well as the M and G proteins are provided by expression plasmids. In other embodiments, such proteins are provided by one or more helper viruses. Such helper viruses can be wild type or mutant. In certain embodiments, the helper virus can be distinguished phenotypically from the virus encoded by the recombinant viral cDNA. For example, it may be desirable to provide monoclonal antibodies that react immunologically with the helper virus but not the virus encoded by the recombinant viral cDNA. Such antibodies can be neutralizing antibodies. In some embodiments, the antibodies can be used in affinity chromatography to separate the helper virus from the recombinant virus. To aid the procurement of such antibodies, mutations can be introduced into the viral cDNA to provide antigenic diversity from the helper virus, such as in a glycoprotein gene.

A recombinant viral genome or antigenome may be constructed for use in the present invention by, e.g., assembling cloned cDNA segments, representing in aggregate the complete genome or antigenome, by polymerase chain reaction or the like (PCR; described in, e.g., U.S. Pat. Nos. 4,683,195 and 4,683,202, and PCR Protocols: A Guide to Methods and Applications, Innis et al., eds., Academic Press, San Diego, 1990) of reverse-transcribed copies of viral mRNA or genome RNA. For example, a first construct may be generated which comprises cDNAs containing the left hand end of the antigenome, spanning from an appropriate promoter (e.g., T7, T3, or SP6 RNA polymerase promoter) and assembled in an appropriate expression vector (such as a plasmid, cosmid, phage, or DNA virus vector). The vector may be modified by mutagenesis and/or insertion of a synthetic polylinker containing unique restriction sites designed to facilitate assembly. The right hand end of the antigenome plasmid may contain additional sequences as desired, such as a flanking ribozyme and single or tandem T7 transcriptional terminators. The ribozyme can be hammerhead type, which would yield a 3′ end containing a single nonviral nucleotide, or can be any of the other suitable ribozymes such as that of hepatitis delta virus (Perrotta et al., Nature 350:434-436, 1991) that would yield a 3′ end free of non-viral nucleotides.

Alternative means to construct cDNA encoding the viral genome or antigenome include reverse transcription-PCR using improved PCR conditions (e.g., as described in Cheng et al., Proc. Natl. Acad. Sci. USA 91:5695-5699, 1994, incorporated herein by reference) to reduce the number of subunit cDNA components to as few as one or two pieces. In other embodiments different promoters can be used (e.g., T3 or SPQ). Different DNA vectors (e.g., cosmids) can be used for propagation to better accommodate the larger size genome or antigenome.

As noted above, defined mutations can be introduced into an infectious viral clone by a variety of conventional techniques (e.g., site-directed mutagenesis) into a cDNA copy of the genome or antigenome. The use of genomic or antigenomic cDNA subfragments to assemble a complete genome or antigenome cDNA as described herein has the advantage that each region can be manipulated separately, where small cDNA constructs provide for better ease of manipulation than large cDNA constructs, and then readily assembled into a complete cDNA.

Certain of the attenuated viruses of the invention will be constructed or modified to limit the growth potential, replication competence, or infectivity of the recombinant virus. Such attenuated viruses and subviral particles are useful as vectors and immunogens, but do not pose certain risks that would otherwise attend administration of a fully infectious (i.e., having approximately a wild-type level of growth and/or replication competence) virus to a host. By attenuated, it is meant a virus or subviral particle that is limited in its ability to grow or replicate in a host cell or a mammalian subject, or is otherwise defective in its ability to infect and/or propagate in or between cells. By way of example, ΔG and G stem are attenuated viruses that are propagation-defective, but replication competent. Often, attenuated viruses and subviral particles will be employed as “vectors”, as described in detail herein below.

Thus, various methods and compositions are provided for producing attenuated VSV particles. In more detailed embodiments, the attenuated virus will exhibit growth, replication and/or infectivity characteristics that are substantially impaired in comparison to growth, replication and/or infectivity of a corresponding wild-type or parental virus. In this context, growth, replication, and/or infectivity may be impaired in vitro and/or in vivo by at least approximately 10-20%, 20-50%, 50-75% and up to 95% or greater compared to wild-type or parental growth, replication and/or infectivity levels.

In some embodiments, viruses with varying degrees of growth or replication defects may be rescued using a combined heat shock/T7-plasmid rescue system described in detail below. Exemplary strains include highly attenuated strains of VSV that incorporate modifications as described below (e.g., a C-terminal G protein truncation, or translocated genes) (see, e.g., Johnson et al., J. Virol. 71:5060-5078, 1997, Schnell et al., Proc. Natl. Acad. Sci. USA 93:11359-11365, 1996; Schnell et al., Cell 90:849-857, 1997; Roberts et al., J. Virol. 72:4704-4711, 1998; and Rose et al., Cell 106:539-549, 2001, each incorporated herein by reference).

Further examples of attenuated viruses are described in further detail below. The attenuated viruses are useful as “vectors”, e.g., by incorporation of a heterologous antigenic determinant into a recombinant vector genome or antigenome. In specific examples, a measles virus (MV) or human immunodeficiency virus (HIV) glycoprotein, glycoprotein domain, or one or more antigenic determinant(s) is incorporated into a VSV vector or “backbone”.

For ease of preparation the N, P, L, M and G viral proteins can be assembled in one or more separate vectors. Many suitable expression vectors are known in the art which are useful for incorporating and directing expression of polynucleotides encoding the RNA polymerase and support proteins, including for example plasmid, cosmid, or phage vectors, defective viral vectors, so-called “replicons” (e.g. sindbis or Venezuelan equine encephalitis replicons) and other vectors useful for directing transient and/or constitutive expression. Transient expression of the RNA polymerase and, where applicable, the N, P, L, M and G proteins, is directed by a transient expression control element operably integrated with the polymerase and/or support vector(s). In one exemplary embodiment, the transient expression control element for the RNA polymerase is an RNA polymerase II regulatory region, as exemplified by the immediate-early human cytomegalovirus [hCMV] promoter and enhancer (see, e.g., U.S. Pat. No. 5,168,062). In other exemplary embodiments, the transient expression control elements for one or more of the N, P, L, M and G proteins is a DNA-dependent RNA polymerase promoter, such as the T7 promoter.

The vectors encoding the viral cDNA, the transiently-expressed RNA polymerase, and the N, P, L, M and G proteins may be introduced into appropriate host cells by any of a variety of methods known in the art, including transfection, electroporation, mechanical insertion, transduction or the like. In some preferred embodiments, the subject vectors are introduced into the cells by electroporation. In other embodiments, the subject vectors are introduced into cultured cells by calcium phosphate-mediated transfection (Wigler et al., Cell 14:725, 1978; Corsaro et al., Somatic Cell Genetics 7:603, 1981; Graham et al., Virology 52:456, 1973), electroporation (Neumann et al., EMBO J. 1:841-845, 1982), DEAE-dextran mediated transfection (Ausubel et al., (ed.) Current Protocols in Molecular Biology, John Wiley and Sons, Inc., NY, 1987), or cationic lipid-mediated transfection (Hawley-Nelson et al., Focus 15:73-79, 1993). In alternate embodiments, a transfection facilitating reagent is added to increase DNA uptake by cells. Many of these reagents are known in the art. LIPOFECTACE® (Life Technologies, Gaithersburg, Md.) and EFFECTENE® (Qiagen, Valencia, Calif.) are common examples. These reagents are cationic lipids that coat DNA and enhance DNA uptake by cells. LIPOFECTACE® forms a liposome that surrounds the DNA while EFFECTINE® coats the DNA but does not form a liposome. Another useful commercial reagent to facilitate DNA uptake is LIPOFECTAMINE-2000® (Invitrogen, Carlsbad, Calif.).

Suitable host cells for use within the invention are capable of supporting a productive infection of the subject attenuated VSV, and will permit expression of the requisite vectors and their encoded products necessary to support viral production. Examples of host cells for use in the methods of the present invention are described in further detail below.

Within the methods and compositions provided herein, coordinate introduction of the RNA polymerase vector, viral cDNA clone, and support vector(s) (e.g., plasmid(s) encoding N, P, L, M and G proteins) into a host cell will be simultaneous. For example, all of the subject DNAs may be combined in a single DNA transfection (e.g., electroporation) mixture and added to a host cell culture simultaneously to achieve coordinate transfection. In alternate embodiments separate transfections may be performed for any two or more of the subject polymerase and support vectors and the viral cDNA vector. Typically, separate transfections will be conducted in close temporal sequence to coordinately introduce the polymerase and support vectors and viral cDNA vector in an effective cotransfection procedure. In one such coordinate transfection protocol, the viral cDNA and/or N, P, L, M and G support plasmid(s) is/are introduced into the host cell prior to transfection of the RNA polymerase plasmid. In other embodiments, the viral cDNA and/or the N, P, L, M and P support plasmid(s) is/are introduced into the host cell simultaneous with or following transfection of the RNA polymerase plasmid into the cell, but before substantial expression of the RNA polymerase begins (e.g., before detectable levels of a T7 polymerase have accumulated, or before levels of T7 sufficient to activate expression of plasmids driven by a T7 promoter have accumulated) in the host cell.

In some embodiments, the method for producing the infectious, attenuated RNA virus may involve an additional heat shock treatment of the host cell to increase recovery of the recombinant virus. After one or more of the viral cDNA expression vectors and the one or more transient expression vectors encoding the RNA polymerase, N protein, P protein, L protein, M protein and G protein are introduced into the host cell, the host cell may be exposed to an effective heat shock stimulus that increases recovery of the recombinant virus.

In one such method, the host cell is exposed to an effective heat shock temperature for a time period sufficient to effectuate heat shock of the cells, which in turn stimulates enhanced viral recovery. An effective heat shock temperature is a temperature above the accepted, recommended or optimal temperature considered in the art for performing rescue of the subject virus. In many instances, an effective heat shock temperature is above 37° C. When a modified rescue method of the invention is carried out at an effective heat shock temperature, there results an increase in recovery of the desired recombinant virus over the level of recovery of recombinant virus when rescue is performed in the absence of the increase in temperature. The effective heat shock temperature and exposure time may vary based upon the rescue system used. Such temperature and time variances can result from differences in the virus selected or host cell type.

Although the temperature may vary, an effective heat shock temperature can be readily ascertained by conducting several test rescue procedures with a particular recombinant virus, and establishing a rate percentage of recovery of the desired recombinant virus as temperature and time of exposure are varied. Certainly, the upper end of any temperature range for performing rescue is the temperature at which the components of the transfection are destroyed or their ability to function in the transfection is depleted or diminished. Exemplary effective heat shock temperature ranges for use within this aspect of the invention are: from about 37° C. to about 50° C., from about 38° C. to about 50° C., from about 39° C. to about 49° C., from about 39° C. to about 48° C., from about 40° C. to about 47° C., from about 41° C. to about 47° C., from about 41° C. to about 46° C. Often, the selected effective heat shock temperature range will be from about 42° C. to about 46° C. In more specific embodiments, effective heat shock temperatures of about 43° C., 44° C., 45° C. or 46° C. are employed.

In conducting the tests to establish a selected effective heat shock temperature or temperature range, one can also select an effective time period for conducting the heat shock procedure. A sufficient time for applying the effective heat shock temperature is a time over which there is a detectable increase in recovery of the desired recombinant virus over the level of recovery of recombinant virus when rescue is performed in the absence of an increase in temperature as noted above. The effective heat shock period may vary based upon the rescue system, including the selected virus and host cell. Although the time may vary, the amount of time for applying an effective heat shock temperature can be readily ascertained by conducting several test rescue procedures with a particular recombinant virus, and establishing a rate or percentage of recovery of the desired recombinant virus as temperature and time are varied. The upper limit for any time variable used in performing rescue is the amount of time at which the components of the transfection are destroyed or their ability to function in the transfection is depleted or diminished. The amount of time for the heat shock procedure may vary from several minutes to several hours, as long as the desired increase in recovery of recombinant virus is obtained. Exemplary effective heat shock periods for use within this aspect of the invention, in minutes, are: from about 5 to about 500 minutes, from about 5 to about 200 minutes, from about 15 to about 300, from about 15 to about 240, from about 20 to about 200, from about 20 to about 150. Often, the effective heat shock period will be from about 30 minutes to about 150 minutes.

Numerous means can be employed to determine the level of improved recovery of a recombinant, attenuated VSV through exposure of host cells to effective heat shock. For example, a chloramphenicol acetyl transferase (CAT) reporter gene can be used to monitor rescue of the recombinant virus according to known methods. The corresponding activity of the reporter gene establishes the baseline and improved level of expression of the recombinant virus. Other methods include detecting the number of plaques of recombinant virus obtained and verifying production of the rescued virus by sequencing. One exemplary method for determining improved recovery involves preparing a number of identically transfected cell cultures and exposing them to different conditions of heat shock (time and temperature variable), and then comparing recovery values for these cultures to corresponding values for control cells (e.g., cells transfected and maintained at a constant temperature of 37° C.). After 72 hours post-transfection, the transfected cells are transferred to a 10 cm plate containing a monolayer of about 75% confluent Vero cells (or cell type of choice for determining plaque formation of the recombinant virus) and continuing incubation until plaques are visible. Thereafter, the plaques are counted and compared with the values obtained from control cells. Optimal heat shock conditions should maximize the number of plaques.

According to these embodiments of the invention, improved viral recovery will be at least about 10% or 25%, and often at least about 40%. In certain embodiments, the increase in the recombinant virus recovered attributed to effective heat shock exposure is reflected by a 2-fold, 5-fold, and up to 10-fold or greater increase in the amount of recombinant virus observed or recovered.

### Plaque Expansion Procedure

In some embodiments of the invention, the host cell in which the viral cDNA, RNA polymerase vector and one or more vector(s) encoding support proteins have been introduced, is subjected to a “plaque expansion” step. This procedure is typically conducted after a period of time (e.g., post-transfection) sufficient to permit expression of the viral cDNA expression vector and one or more expression vectors that encode(s) and direct(s) transient expression of the RNA polymerase, N protein, P protein, L protein, M protein and G protein. To achieve plaque expansion, the host cell, which often has become impaired in its ability to support further viral expansion, is co-cultured with a plaque expansion cell of the same or different cell type. This co-culture step allows spread of rescued virus to the plaque expansion cell, which is more amenable to vigorous expansion of the virus. Typically, a culture of host cells is transferred onto one or more layer(s) of plaque expansion cells. For example, a culture of host cells can be spread onto a monolayer of plaque expansion cells and the attenuated VSV will thereafter infect the plaque expansion cells and expand further therein. In some embodiments, the host cell is of the same, or different, cell type as the plaque expansion cell.

In certain embodiments, both the host cells used for viral rescue, as well as the plaque expansion cells transiently express an optimized VSV G protein coding sequence. In other embodiments, the host cells used for viral rescue may express a functional, but non-optimized G coding sequence (e.g., a native G coding sequence), provided that the plaque expansion cells, which are to be infected with the rescued virus during the co-culture step, express the optimized VSV G coding sequence, either transiently or constitutively. In some embodiments, expression of VSV G protein from an optimized VSV G sequence in the plaque expansion cells is under the control of a cytomegalovirus-derived RNA polymerase II promoter.

The plaque expansion methods and compositions of the invention provide improved rescue methods for producing attenuated VSV, such as including, but not limited to, propagation-defective VSV. Typically, the viral rescue method entails transfecting a host cell with: a viral cDNA expression vector comprising an isolated nucleic acid molecule encoding a genome or antigenome of an attenuated VSV; expression vector encoding and directing expression of an RNA polymerase, along with an expression vector which comprises a nucleic acid molecule encoding a functional G protein (e.g., a non-optimized or optimized VSV G gene). The viral rescue method further includes introducing into the host cell one or more other support expression vectors which comprise at least one isolated nucleic acid molecule encoding trans-acting proteins necessary for encapsidation, transcription and replication (i.e., N, P and L proteins of VSV). The viral rescue method may further include transfecting the cells with a support vector encoding an M protein of VSV for a productive infection. The vectors are introduced into the host cell under conditions sufficient to permit co-expression of said vectors and production of the attenuated, mature virus particles.

The attenuated VSV is rescued and the rescued material is then preferably co-cultured with plaque expansion cells. This allows spread of the rescued virus to the plaque expansion cell via infection. The plaque expansion cell is more amenable to vigorous expansion of the virus. The attenuated VSV may then be recovered from the co-culture. In some embodiments, the viral rescue cells are transferred onto at least one layer of plaque expansion cells that have been transiently transfected with a plasmid containing an optimized VSV G gene, or that constitutively express VSV G protein encoded by an optimized VSV G gene.

In order to achieve plaque expansion, the transfected cells are typically transferred to co-culture containers of plaque expansion cells. Any of the various plates or containers known in the art can be employed for the plaque expansion step. In certain embodiments, the transfected cells are transferred onto a monolayer of plaque expansion cells that is at least about 50% confluent. Alternatively, the plaque expansion cells are at least about 60% confluent, or even at least about 75% confluent. In certain embodiments, the surface area of the plaque expansion cells is greater than the surface area used for preparing the transfected virus. An enhanced surface area ratio of from 2:1 to 100:1 can be employed as desired. An enhanced surface area of at least 10:1 is often desired.

### Optimized VSV G Gene

Propagation-defective viruses offer clear safety advantages for use in humans. These vectors are restricted to a single round of replication and are unable to spread beyond primary infected cells. One such vector, which is described in detail below, has the entire G gene deleted (ΔG), and therefore requires G protein transcomplementation for propagation of infectious virus particles in vitro. Another vector, which is described in detail below, has most of the G protein ectodomain deleted (Gstem), retaining the cytoplasmic tail (CT) region, transmembrane domain, and 42 amino acids of the membrane proximal ectodomain. This vector is also propagation-defective, requiring G protein in trans for production of infectious particles in vitro.

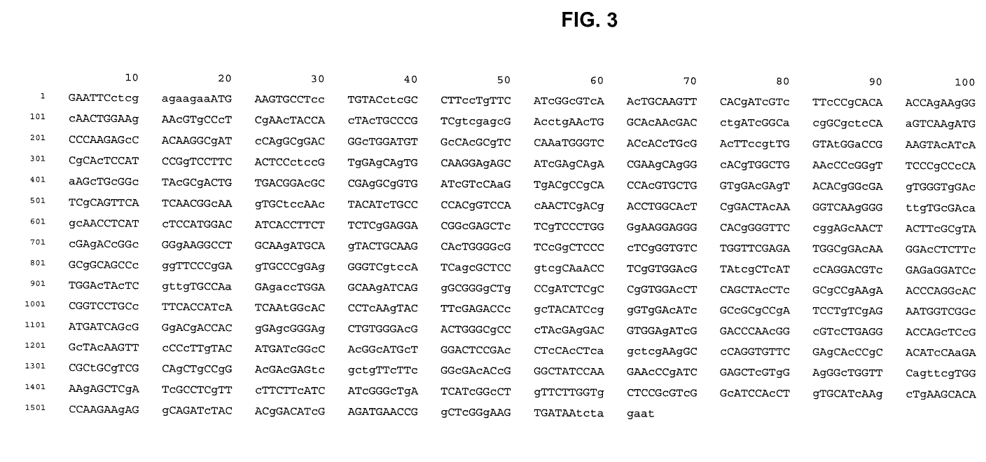

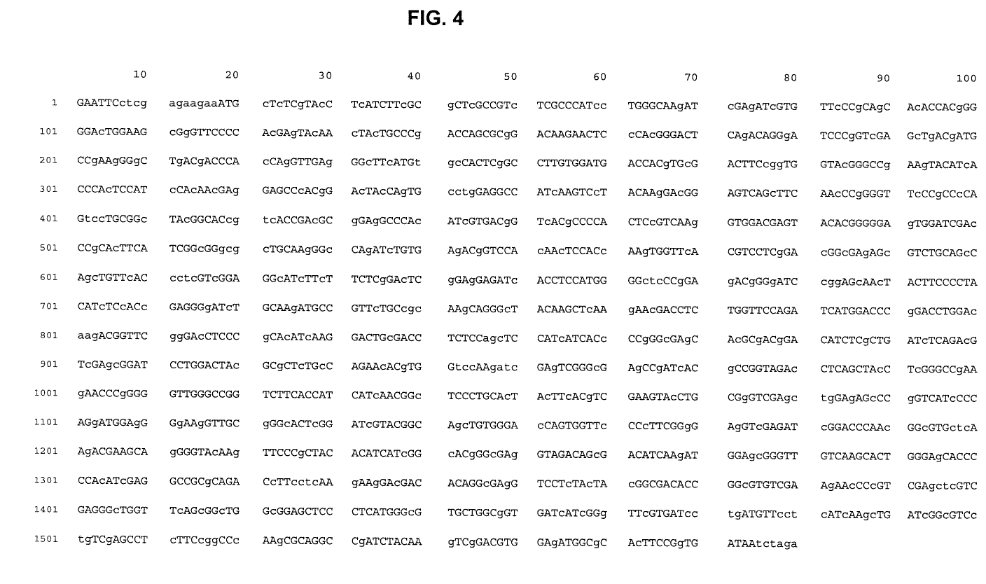

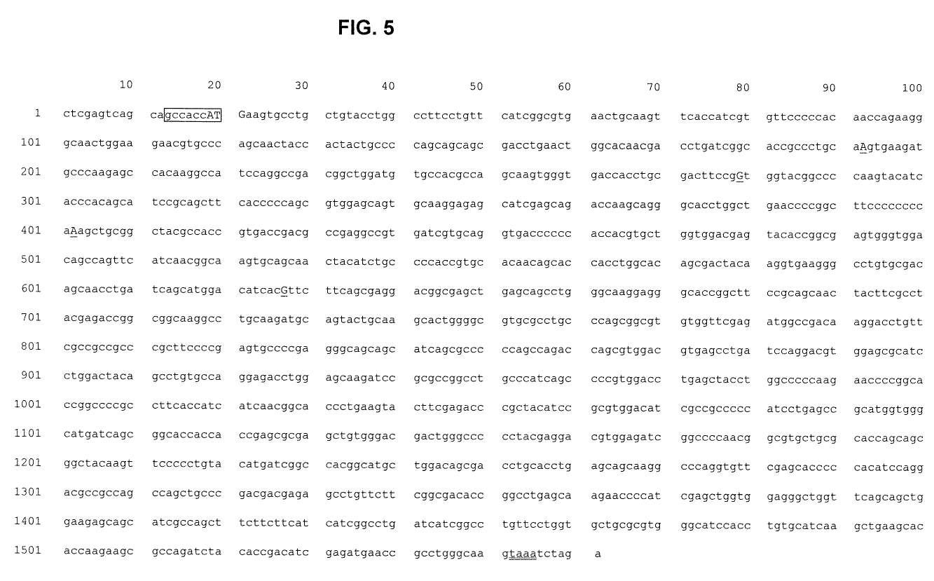

Although propagation-defective viruses have been known to offer safety advantages, prior to the present invention, there were difficulties in providing adequate quantities of complementing G protein to allow efficient vector amplification during industrial scale manufacture. As detailed in the Examples, extensive studies were conducted by the present inventors to identify conditions that support maximal G protein expression. Two methods of coding sequence optimization were analyzed to determine if they might improve transient expression of VSV G protein. One method, described as RNA optimization (RNAopt) and used synonymous nucleotide substitutions to increase GC content and disrupt sequence motifs that inhibit nuclear export, decrease translation, or destabilize mRNAs (Schneider, et al. J Virol 71:4892-903, 1997); Schwartz, et al. J Virol 66:7176-82, 1992; Schwartz, et al. J Virol 66:150-9, 1992). VSV G (RNA optimized) coding sequences for Indiana and New Jersey serotypes are shown, for example, in FIG. 3 (SEQ ID NO. 4) and FIG. 4 (SEQ ID NO. 5), respectively, where lower case letters indicated substitutions made during optimization. The second method of optimization is a codon optimization method detailed below in Table 1 (Opt-1). A VSV G coding sequence (Indiana serotype) obtained using the codon optimization method is shown, for example, in FIG. 5 (SEQ ID NO. 3).

TABLE 1
Optimization Method 1 (Opt-1)

| | |
|---|---|
| Step 1 | Generate a G coding sequence composed of high frequency human |
| | codons. |
| | Reverse translation of VSV-Gin amino acid sequence was performed with |
| | the Backtranslate program in the SeqWeb software suite (Accelrys |
| | Software, Inc). |
| Step 2 | Introduce synonymous base substitutions that disrupt predicted mRNA |
| | splicing signals. |
| | Splice site predictions were made using an internet tool available through |
| | the Berkeley Drosophila Genome Project at www.fruitfly.org: (Reese, et al. |
| | J Comput Biol 4: 311-23, 1997) |
| Step 3 | Place the translation initiation codon in a favorable context as described |
| | by Kozak (Kozak. J Biol Chem 266: 19867-70, 1991) |
| Step 4 | Place translation termination signal in a favorable context (Kochetov, et al. |
| | FEBS Lett 440: 351-5, 1998) |

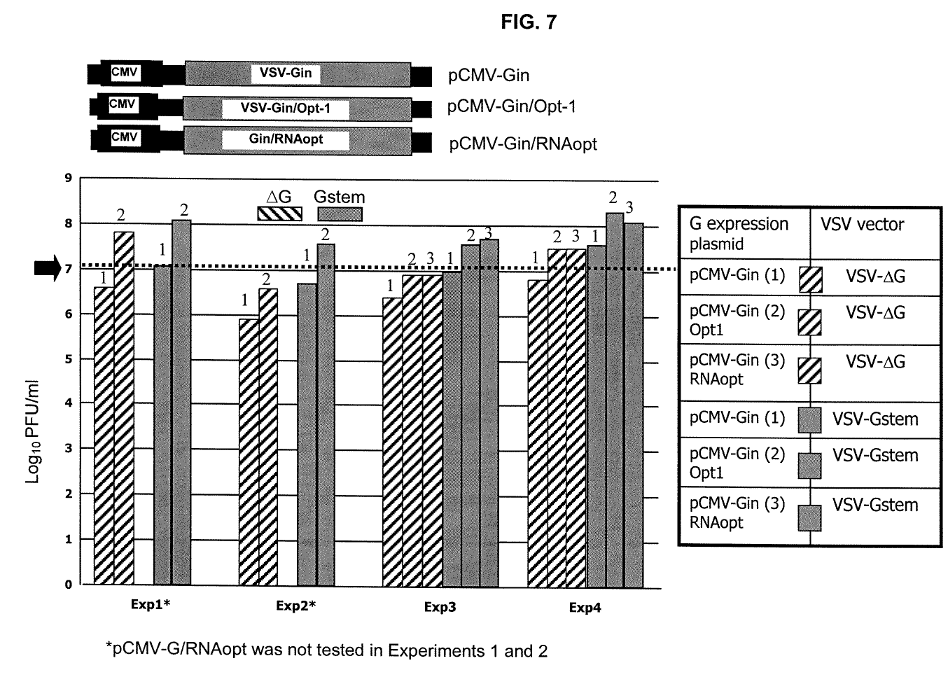

As described in further detail in the Examples, it was discovered that electroporation of plasmids containing optimized VSV G coding sequences produced higher levels of G protein expression in Vero cells as compared to the native Gin open reading frame. Thereafter, studies were conducted to determine whether the increased abundance of G enhanced packaging yields of propagation-defective vectors. As described in further detail in the Examples and in FIG. 7, the results indicated that both plasmids containing optimized VSV G coding sequences (pCMV-Gin/Opt1 and pCMV-Gin/RNAopt) promoted more efficient packaging as compared with the plasmid containing the native Gin open reading frame (pCMV-Gin).

In some embodiments, an optimized VSV G gene is selected from the following: SEQ ID NO: 3, SEQ ID NO: 4, and SEQ ID NO: 5.

### Cells

### 1. Viral Rescue Cells

Host cells used for viral rescue can be selected from a prokaryotic cell or a eukaryotic cell. Suitable cells include insect cells such as Sf9 and Sf21, bacterial cells with an appropriate promoter such as E. coli, and yeast cells such as S. cerevisiae. Host cells are typically chosen from vertebrate, e.g., primate, cells. Typically, a cell line is employed that yields a detectable cytopathic effect in order that rescue of viable virus may be easily detected. Often, the host cells are derived from a human cell, such as a human embryonic kidney (HEK) cell. Vero cells (African green monkey kidney cells), as well as many other types of cells can also be used as host cells. In some exemplary embodiments, Vero cells are used as host cells. In the case of VSV, the transfected cells are grown on Vero cells because the virus spreads rapidly on Vero cells and makes easily detectable plaques. Moreover, Vero cells are qualified for production for human administration. The following are examples of other suitable host cells: (1) Human Diploid Primary Cell Lines: e.g. WI-38 and MRC-5 cells; (2) Monkey Diploid Cell Line: e.g. Cos, Fetal Rhesus Lung (FRhL) cells; (3) Quasi-Primary Continuous Cell Line: e.g. AGMK-African green monkey kidney cells; (4) Human 293 cells (qualified) and (5) rodent (e.g., CHO, BHK), canine e.g., Madin-Darby Canine Kidney (MDCK), and primary chick embryo fibroblasts. Exemplary specific cell lines that are useful within the methods and compositions of the invention include HEp-2, HeLa, HEK (e.g., HEK 293), BHK, FRhL-DBS2, LLC-MK2, MRC-5, and Vero cells.

### 2. Plaque Expansion Cells

As described in further detail herein, a method of producing attenuated VSV particles according to the present invention may include growing the host cells used in the rescue of the viral particles with plaque expansion cells. This permits the spread of recovered attenuated VSV particles to the plaque expansion cells. In some embodiments, the plaque expansion cells are of a same or different cell type as the host cells used for viral rescue.

The plaque expansion cells are selected based on the successful growth of the native or recombinant virus in such cells. Often, the host cell employed in conducting the transfection is not an optimal host for growth of the desired recombinant, attenuated virus. The recovery of recombinant, attenuated virus from the transfected cells can therefore be enhanced by selecting a plaque expansion cell in which the native virus or the recombinant virus exhibits enhanced growth. Various plaque expansion cells can be selected for use within this aspect of the invention, in accordance with the foregoing description. Exemplary specific plaque expansion cells that can be used to support recovery and expansion of recombinant, attenuated VSVs of the invention are selected from HEp-2, HeLa, HEK, BHK, FRhL-DBS2, LLC-MK2, MRC-5, and Vero cells. Additional details concerning heat shock and plaque expansion methods for use within the invention are provided in PCT publication WO 99/63064, incorporated herein by reference.

In some embodiments, the plaque expansion cells are transiently transfected with an expression plasmid including an optimized VSV G gene. Thereafter, the transfected cells are typically incubated overnight at 37° C., 5% CO₂ before being used to establish a coculture with the viral rescue cells. The rescued, attenuated virus infects the plaque expansion cells during the coculture step, and the virus expands further therein. In some other embodiments, the plaque expansion cells may constitutively express VSV G protein encoded by an optimized VSV G gene.

### Attenuated Vesicular Stomatitis Viruses

### 1. Truncated G Cytoplasmic Tail (CT) Region

In certain embodiments, an attenuated VSV for use in the present invention expresses a G protein having a truncated cytoplasmic tail (CT) region. For example, it is known in the art that G gene mutations which truncate the carboxy-terminus of the cytoplasmic domain influence VSV budding and attenuate virus production (Schnell, et al. The EMBO Journal 17(5):1289-1296, 1998; Roberts, et al. J Virol, 73:3723-3732, 1999). The cytoplasmic domain of wild-type VSV G protein comprises twenty-nine amino acids (RVGIHLCIKLKHTKKRQIYTDIEMNRLGK-COOH; SEQ ID NO: 6).

In some embodiments, an attenuated VSV expresses a G protein having a cytoplasmic tail region truncated to one amino acid (G-CT1). For example, the attenuated VSV may express a G protein in which the last twenty-eight amino acid residues of the cytoplasmic domain are deleted (retaining only arginine from the twenty-nine amino acid wild-type cytoplasmic domain of SEQ ID NO: 6).

In some other embodiments, an attenuated VSV expresses a G protein having a cytoplasmic tail region truncated to nine amino acids (G-CT-9). For example, the attenuated VSV may express a G protein in which the last twenty carboxy-terminal amino acids of the cytoplasmic domain are deleted (relative to the twenty-nine amino acid wild-type cytoplasmic domain of SEQ ID NO: 6).

### 2. G Gene Deletions

In some embodiments, an attenuated VSV lacks a VSV G protein (VSV-ΔG). For example, an attenuated VSV of the invention may be a virus in which a VSV G gene) is deleted from the genome. In this regard, Roberts, et al. described a VSV vector in which the entire gene encoding the G protein was deleted (ΔG) and substituted with influenza haemagglutinin (HA) protein, wherein the VSV vector (ΔG-HA) demonstrated attenuated pathogenesis (Roberts, et al. Journal of Virology, 73:3723-3732, 1999).

### 3. G-Stem Mutations

In some other embodiments, an attenuated VSV expresses a G protein having a truncated extracellular domain (VSV-Gstem). For example, an attenuated VSV of the invention may include a mutation in the G gene, wherein the encoded G protein has a mutation in the membrane-proximal stem region of the G protein ectodomain, referred to as G-stem protein. The G-stem region comprises amino acid residues 421-462 of the G protein. Prior studies have demonstrated the attenuation of VSV via insertion and/or deletion (e.g., truncation) mutations in the G-stem of the G protein (Robison and Whitt, J Virol 74 (5):2239-2246, 2000; Jeetendra, et al., J Virol 76(23):12300-11, 2002; Jeetendra, et al., J Virol 77 (23):12807-18, 2003).

In some embodiments, the attenuated VSV is one in which the G coding sequence is replaced with a modified version that encodes only 18 amino-terminal residues of the signal sequence fused to the C-terminal 91 amino acids of G of which approximately 42 residues from a truncated extracellular domain (G-stem). This type of G gene modification may be constructed using the method of Robison and Whitt, J Virol 74 (5):2239-2246, 2000.

### 4. Gene Shuffling Mutations

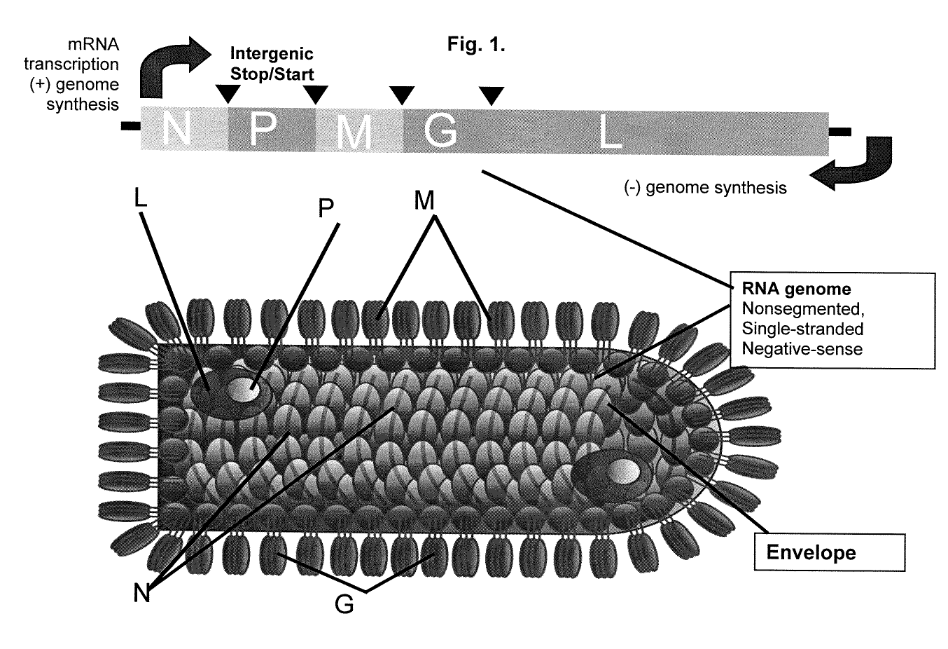

In certain embodiments, an attenuated VSV of the invention comprises a gene shuffling mutation in its genome. As defined herein, the terms “gene shuffling”, “shuffled gene”, “shuffled”, “shuffling”, “gene rearrangement” and “gene translocation” may be used interchangeably and refer to a change (mutation) in the order of the wild-type VSV genome. As defined herein, a wild-type VSV genome has the following gene order, which is depicted in FIG. 1: 3′-NPMGL-5′.

It is known in the art, that the position of a VSV gene relative to the 3′ promoter determines the level of expression and virus attenuation (U.S. Pat. No. 6,596,529 to Wertz, et al. and Wertz et al., Proc. Natl. Acad. Sci USA 95:3501-6, 1998, each specifically incorporated herein by reference). There is a gradient of expression, with genes proximal to the 3′ promoter expressed more abundantly than genes distal to the 3′ promoter. The nucleotide sequences encoding VSV G, M, N, P and L proteins are known in the art (Rose and Gallione, J Virol 39:519-528, 1981; Gallione et al., J Virol 39:529-535, 1981). For example, U.S. Pat. No. 6,596,529 describes gene shuffling mutations in which the gene for the N protein is translocated (shuffled) from its wild-type promoter-proximal first position to successively more distal positions on the genome, in order to successively reduce N protein expression (e.g., 3′-PNMGL-5′,3′-PMNGL-5′,3′-PMGNL-5′, referred to as N2, N3 and N4, respectively). Positionally-shifted VSV mutants are also described in, for e.g., U.S. Pat. No. 6,136,585 to Ball, et al.

Thus, in certain embodiments, an attenuated VSV comprises a gene shuffling mutation in its genome. A gene shuffling mutation may comprise a translocation of the N gene (e.g., 3′-PNMGL-5′ or 3′-PMNGL-5′). For example, in some embodiments, the attenuated VSV comprises the N gene, which has been translocated downstream from its wild-type position in the viral genome, thereby resulting in a reduction in N protein expression.

It should be noted herein, that the insertion of a foreign nucleic acid sequence (e.g., HIV gag) into the VSV genome 3′ to any of the N, P, M, G or L genes, effectively results in a “gene shuffling mutation” as defined above. For example, when the HIV gag gene is inserted into the VSV genome at position one (e.g., 3′-gag₁-NPMGL-5′), the N, P, M, G and L genes are each moved from their wild-type positions to more distal positions on the genome. Thus, in certain embodiments of the invention, a gene shuffling mutation includes the insertion of a foreign nucleic acid sequence into the VSV genome 3′ to any of the N, P, M, G or L genes (e.g., 3′-gag₁-NPMGL-5′, 3′-N-gag₂-PMGL-5′,3′-NP-gag₃-MGL-5′, etc.).

### 5. Non-Cytopathic M Gene Mutations

In certain other embodiments, an attenuated VSV of the invention includes a non-cytopathic mutation (Mncp) in the M gene. The VSV (Indiana serotype) M gene encodes a 229 amino acid M (matrix) protein.

It is known in the art that the M mRNA further encodes two additional proteins, referred to as M2 and M3 (Jayakar and Whitt, J Virol 76(16):8011-8018 2002). The M2 and M3 proteins are synthesized from downstream methionines in the same reading frame that encodes the 229 amino acid M protein (referred to as M1), and lack the first thirty-two (M2 protein) or fifty (M3 protein) amino acids of the M1 protein. It has been observed that cells infected with a recombinant VSV that expresses the M protein, but not M2 and M3, exhibit a delayed onset of cytopathic effect (in certain cell types), yet produce a normal virus yield.

Thus, in certain embodiments, an attenuated VSV of the invention includes a non-cytopathic mutation in the M gene, wherein the M gene mutation reduces the expression of two overlapping in-frame polypeptides that are expressed from the M protein mRNA by initiation of protein synthesis at internal AUGs. Such an M gene mutation results in a virus that does not express the M2 or M3 protein. These mutations also affect IFN induction, nuclear transport and other functions. See, for example, Jayakar and Whitt, J Virol 76(16):8011-8018, 2002.

### Heterologous Antigens

In some embodiments, the attenuated VSV expresses a heterologous antigen, so that the VSV serves as a vector. For example, in certain embodiments, the attenuated VSV may include a foreign RNA sequence as a separate transcriptional unit inserted into or replacing a site of the genome nonessential for replication, wherein the foreign RNA sequence (which is in the negative sense) directs the production of a protein capable of being expressed in a host cell infected by VSV. This recombinant genome is originally produced by insertion of foreign DNA encoding the protein into the VSV cDNA. In certain embodiments, any DNA sequence which encodes an immunogenic antigen, which produces prophylactic or therapeutic immunity against a disease or disorder, when expressed as a fusion or non-fusion protein in an attenuated VSV of the invention, alone or in combination with other antigens expressed by the same or a different VSV, is isolated and incorporated in the VSV vector for use in the immunogenic compositions of the present invention.

In certain embodiments, expression of an antigen by an attenuated recombinant VSV induces an immune response against a pathogenic microorganism. For example, an antigen may display the immunogenicity or antigenicity of an antigen found on bacteria, parasites, viruses, or fungi which are causative agents of diseases or disorders. In one embodiment, antigens displaying the antigenicity or immunogenicity of an antigen of a human pathogen or other antigens of interest are used.

In some embodiments, the heterologous antigen encoded by the attenuated VSV is selected from one or more of the following: measles virus, subgroup A and subgroup B respiratory syncytial viruses, human parainfluenza viruses, mumps virus, human papilloma viruses of type 1 or type 2, human immunodeficiency viruses, herpes simplex viruses, cytomegalovirus, rabies virus, human metapneumovirus, Epstein Barr virus, filoviruses, bunyaviruses, flaviviruses, alphaviruses, influenza viruses, hepatitis C virus and C. trachomatis.

To determine immunogenicity or antigenicity by detecting binding to antibody, various immunoassays known in the art are used, including but not limited to, competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, neutralization assays, etc. In one embodiment, antibody binding is measured by detecting a label on the primary antibody. In another embodiment, the primary antibody is detected by measuring binding of a secondary antibody or reagent to the primary antibody. In a further embodiment, the secondary antibody is labeled. Many means are known in the art for detecting binding in an immunoassay. In one embodiment for detecting immunogenicity, T cell-mediated responses are assayed by standard methods, e.g., in vitro or in vivo cytotoxicity assays, tetramer assays, elispot assays or in vivo delayed-type hypersensitivity assays.

Parasites and bacteria expressing epitopes (antigenic determinants) that are expressed by an attenuated VSV (wherein the foreign RNA directs the production of an antigen of the parasite or bacteria or a derivative thereof containing an epitope thereof) include but are not limited to those listed in Table 2.

TABLE 2
PARASITES AND BACTERIA EXPRESSING EPITOPES
THAT CAN BE EXPRESSED BY VSV

PARASITES
Plasmodium spp.
Eimeria spp.
nematodes
Schistosoma spp.
Leishmania spp.
BACTERIA
Vibrio cholerae
Streptococcus pneumoniae
Streptococcus pyogenes
Streptococcus agalactiae
Staphylococcus aureus
Staphylococcus epidermidis
Neisseria meningitidis
Neisseria gonorrhoeae
Corynebacterium diphtheriae
Clostridium tetani
Bordetella pertussis
Haemophilus spp. (e.g., influenzae)
Chlamydia spp.
Enterotoxigenic Escherichia coli
Helicobacter pylori
mycobacteria

In another embodiment, the antigen comprises an epitope of an antigen of a nematode, to protect against disorders caused by such worms. In another embodiment, any DNA sequence which encodes a Plasmodium epitope, which when expressed by a recombinant VSV, is immunogenic in a vertebrate host, is isolated for insertion into VSV (−) DNA according to the present invention. The species of Plasmodium which serve as DNA sources include, but are not limited to, the human malaria parasites P. falciparum, P. malariae, P. ovale, P. vivax, and the animal malaria parasites P. berghei, P. yoelii, P. knowlesi, and P. cynomolgi. In yet another embodiment, the antigen comprises a peptide of the β-subunit of Cholera toxin.

Viruses expressing epitopes that are expressed by an attenuated VSV (wherein the foreign RNA directs the production of an antigen of the virus or a derivative thereof comprising an epitope thereof) include, but are not limited to, those listed in Table 3, which lists such viruses by family for purposes of convenience and not limitation.

TABLE 3
VIRUSES EXPRESSING EPITOPES THAT CAN BE
EXPRESSED BY VSV

I. Picornaviridae
Enteroviruses
Poliovirus
Coxsackievirus
Echovirus
Rhinoviruses
Hepatitis A Virus
II. Caliciviridae
Norwalk group of viruses
III. Togaviridae and Flaviviridae
Togaviruses (e.g., Dengue virus)
Alphaviruses
Flaviviruses (e.g., Hepatitis C virus)
Rubella virus
IV. Coronaviridae
Coronaviruses
V. Rhabdoviridae
Rabies virus
VI. Filoviridae
Marburg viruses
Ebola viruses
VII. Paramyxoviridae
Parainfluenza virus
Mumps virus
Measles virus
Respiratory syncytial virus
Metapneumovirus
VIII. Orthomyxoviridae
Orthomyxoviruses (e.g., Influenza virus)
IX. Bunyaviridae
Bunyaviruses
X. Arenaviridae
Arenaviruses
XI. Reoviridae
Reoviruses
Rotaviruses
Orbiviruses
XII. Retroviridae
Human T Cell Leukemia Virus type I
Human T Cell Leukemia Virus type II
Human Immunodeficiency Viruses (e.g., type
I and type II
Simian Immunodeficiency Virus
Lentiviruses
XIII. Papovaviridae
Polyomaviruses
Papillomaviruses
XIV. Parvoviridae
Parvoviruses
XV. Herpesviridae
Herpes Simplex Viruses
Epstein-Barr virus
Cytomegalovirus
Varicella-Zoster virus
Human Herpesvirus-6
human herpesvirus-7
Cercopithecine Herpes Virus 1 (B virus)
XVI. Poxviridae
Poxviruses
XVIII. Hepadnaviridae
Hepatitis B virus
XIX. Adenoviridae

In specific embodiments, the antigen encoded by the foreign sequences that is expressed upon infection of a host by the attenuated VSV, displays the antigenicity or immunogenicity of an influenza virus hemagglutinin; human respiratory syncytial virus G glycoprotein (G); measles virus hemagglutinin or herpes simplex virus type-2 glycoprotein gD.

Other antigens that are expressed by attenuated VSV include, but are not limited to, those displaying the antigenicity or immunogenicity of the following antigens: Poliovirus I VP1; envelope glycoproteins of HIV I; Hepatitis B surface antigen; Diphtheria toxin; streptococcus 24M epitope, SpeA, SpeB, SpeC or C5a peptidase; and gonococcal pilin.

In other embodiments, the antigen expressed by the attenuated VSV displays the antigenicity or immunogenicity of pseudorabies virus g50 (gpD), pseudorabies virus II (gpB), pseudorabies virus gill (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydrodysenteriae protective antigen, Bovine Viral Diarrhea glycoprotein 55, Newcastle Disease Virus hemagglutinin-neuraminidase, swine flu hemagglutinin, or swine flu neuraminidase.

In certain embodiments, an antigen expressed by the attenuated VSV displays the antigenicity or immunogenicity of an antigen derived from a canine or feline pathogen, including, but not limited to, feline leukemia virus, canine distemper virus, canine adenovirus, canine parvovirus and the like.

In certain other embodiments, the antigen expressed by the attenuated VSV displays the antigenicity or immunogenicity of an antigen derived from Serpulina hyodysenteriae, Foot and Mouth Disease Virus, Hog Cholera Virus, swine influenza virus, African Swine Fever Virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein 1).

In another embodiment, the antigen displays the antigenicity or immunogenicity of a glycoprotein of La Crosse Virus, Neonatal Calf Diarrhea Virus, Venezuelan Equine Encephalomyelitis Virus, Punta Toro Virus, Murine Leukemia Virus or Mouse Mammary Tumor Virus.

In other embodiments, the antigen displays the antigenicity or immunogenicity of an antigen of a human pathogen, including but not limited to human herpesvirus, herpes simplex virus-1, herpes simplex virus-2, human cytomegalovirus, Epstein-Barr virus, Varicella-Zoster virus, human herpesvirus-6, human herpesvirus-7, human influenza virus, human immunodeficiency virus (type 1 and/or type 2), rabies virus, measles virus, hepatitis B virus, hepatitis C virus, Plasmodium falciparum, and Bordetella pertussis.

Potentially useful antigens or derivatives thereof for use as antigens expressed by attenuated VSV are identified by various criteria, such as the antigen's involvement in neutralization of a pathogen's infectivity, type or group specificity, recognition by patients' antisera or immune cells, and/or the demonstration of protective effects of antisera or immune cells specific for the antigen.

In another embodiment, foreign RNA of the attenuated VSV directs the production of an antigen comprising an epitope, which when the attenuated VSV is introduced into a desired host, induces an immune response that protects against a condition or disorder caused by an entity containing the epitope. For example, the antigen can be a tumor specific antigen or tumor-associated antigen, for induction of a protective immune response against a tumor (e.g., a malignant tumor). Such tumor-specific or tumor-associated antigens include, but are not limited to, KS 1/4 pan-carcinoma antigen; ovarian carcinoma antigen (CA125); prostatic acid phosphate; prostate specific antigen; melanoma-associated antigen p97; melanoma antigen gp75; high molecular weight melanoma antigen and prostate specific membrane antigen.

The foreign DNA encoding the antigen, that is inserted into a non-essential site of the attenuated VSV DNA, optionally further comprises a foreign DNA sequence encoding a cytokine capable of being expressed and stimulating an immune response in a host infected by the attenuated VSV. For example, such cytokines include but are not limited to interleukins 1α, 1β, 2, 4, 5, 6, 7, 8, 10, 12, 13, 14, 15, 16, 17 and 18, interferon-α, interferon-β, interferon-γ, granulocyte colony stimulating factor, granulocyte macrophage colony stimulating factor and the tumor necrosis factors α and β.

### Immunogenic and Pharmaceutical Compositions

In certain embodiments, the invention is directed to an immunogenic composition comprising an immunogenically effective amount of attenuated VSV particles produced according to the methods of the present invention in a pharmaceutically acceptable carrier. In some embodiments, at least one foreign RNA sequence is inserted into or replaces a region of the VSV genome non-essential for replication.

The attenuated VSV particles of the invention are formulated for administration to a mammalian subject (e.g., a human). Such compositions typically comprise the VSV vector and a pharmaceutically acceptable carrier. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the VSV vector, such media are used in the immunogenic compositions of the invention. Supplementary active compounds may also be incorporated into the compositions.

Thus, a VSV immunogenic composition of the invention is formulated to be compatible with its intended route of administration. Examples of routes of administration include parenteral (e.g., intravenous, intradermal, subcutaneous, intramuscular, intraperitoneal) and mucosal (e.g., oral, rectal, intranasal, buccal, vaginal, respiratory). Solutions or suspensions used for parenteral, intradermal, or subcutaneous application include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH is adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (e.g., glycerol, propylene glycol, and liquid polyetheylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid, and the like. In many cases, it is preferable to include isotonic agents, for example, sugars, polyalcohols such as mannitol, sorbitol, sodium chloride in the composition. Prolonged absorption of the injectable compositions is brought about by including in the composition an agent which delays absorption, for example, aluminum monostearate and gelatin.

Sterile injectable solutions are prepared by incorporating the VSV vector in the required amount (or dose) in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant (e.g., a gas such as carbon dioxide, or a nebulizer). Systemic administration can also be by mucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. The compounds are also prepared in the form of suppositories (e.g., with conventional suppository bases such as cocoa butter and other glycerides) or retention enemas for rectal delivery.

In certain embodiments, it is advantageous to formulate oral or parenteral compositions in dosage unit form for ease of administration and uniformity of dosage. Dosage unit form as used hereinafter refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

All patents and publications cited herein are hereby incorporated by reference.

### EXAMPLES

### Example 1

### Preparation of Recombinant DNA

A plasmid vector encoding T7 RNAP (pCMV-T7) was prepared by cloning the polymerase open reading frame (ORF) into pCl-neo (Promega) 3′ of the hCMV immediate-early promoter/enhancer region. Before insertion of the T7 RNAP ORF, pCl-neo was modified to remove the T7 promoter located 5′ of the multiple cloning site, generating vector pCl-neo-Bcl. The T7 RNAP gene was inserted into pCl-Neo-BCl using EcoR I and Xba I restriction sites incorporated into PCR primers used to amplify the T7 RNAP coding sequence. A Kozak (Kozak, J Cell Biol 108, 229-241, 1989) consensus sequence was included 5′ of the initiator ATG to provide an optimal sequence context for translation.

Plasmids encoding VSV N, P, L, M and G polypeptides were prepared by inserting the appropriate ORFs 3′ of the T7 bacteriophage promoter and encephalomyocarditis virus internal ribosome entry site (IRES) (Jang et al., J Virol 62, 2636-2643, 1988; Pelletier and Sonenberg, Nature 334, 320-325, 1988) in plasmid vector pT7 as described by Parks, et al. (Parks, et al. Virus Res 83, 131-147, 2002). The inserted coding sequences are flanked at the 3′ end by a plasmid-encoded poly-A sequence and a T7 RNAP terminator. Plasmids encoding VSV N, P, L, M, and glycoprotein (G) were derived from the Indiana serotype genomic cDNA clone (Lawson, et al., Proc Natl Acad Sci USA 92, 4477-4481, 1995) or the New Jersey serotype clone (Rose, et al. J Virol 74, 10903-10910, 2000).

Expression plasmids encoding VSV native G or VSV optimized G coding sequences controlled by the hCMV promoter/enhancer (pCMV-G or pCMV-Opt1; pCMV-RNAopt, respectively) are described below in Example 2. These plasmids were used to provide the glycoprotein in trans while propagating VSV ΔG or VSV-Gstem vectors. The G protein coding sequences were cloned into the modified pCl-neo vector described above in the present example. The G coding sequence was inserted into the modified pCl-neo vector using Xho I (5′) and Xba I (3′) restriction sites incorporated into PCR primers used to amplify the G coding sequence.

Recombinant VSV genomic clones were prepared using standard cloning procedures (Ausubel, et al., Current Protocols in Molecular Biology. Greene Publishing Associates and Wiley Interscience, New York, 1987) and the Indiana serotype pVSV-XN2 genomic cDNA clone as starting material (Lawson, et al., Proc Natl Acad Sci USA 92, 4477-4481, 1995). Genomic clones lacking the G gene (ΔG) were similar to those described by Roberts, et al. (Roberts, et al. J Virol 73, 3723-3732, 1999). A second type of G gene modification was constructed using the approach of Robison and Whitt (Robison and Whitt, J Virol 74, 2239-2246, 2000) in which the G coding sequence was replaced with a modified version that encodes only 18 amino-terminal (N-terminal) residues of the signal sequence fused to the C-terminal 91 amino acids of which approximately 42 residues forms a truncated extracellular domain (Gstem). In some recombinant VSV constructs, the G protein gene was replaced with the equivalent gene from the New Jersey Serotype (Rose, et al. J Virol 74, 10903-10910, 2000).

### Example 2

### Investigation of Packaging Method Improvements

Investigation of packaging method improvements focused on two key steps in the process: 1) transient protein production driven by plasmid DNA, and 2) the efficiency of virion morphogenesis. In the method described in Example 3 below, modifications were identified that improve the efficiency of these steps, resulting in a procedure that routinely yields over 1×10⁷ IU per ml of Vero cell culture medium.

Studies were conducted to identify conditions that supported maximal G protein expression. Empirical research performed earlier identified electroporation as a method that promoted reproducible and efficient introduction of plasmid DNA into Vero cells (Parks, et al., 2006, Method for the recovery of non-segmented, negative-stranded RNA viruses from cDNA, published United States patent application 20060153870; Witko, et al. J Virol Methods 135:91-101) and subsequent method refinement relied on this finding, because electroporation is a scalable technology (Fratantoni, et al. Cytotherapy 5:208-10, 2003), and because Vero cells are a well characterized cell substrate that has been used for production of a live rotavirus vaccine (Merck, RotaTeq (Rotavirus Vaccine, Live, Oral, Pentavalent) FDA. Online, 2006 posting date; Sheets, R. (History and characterization of the Vero cell line) FDA. Online, 2000 posting date).

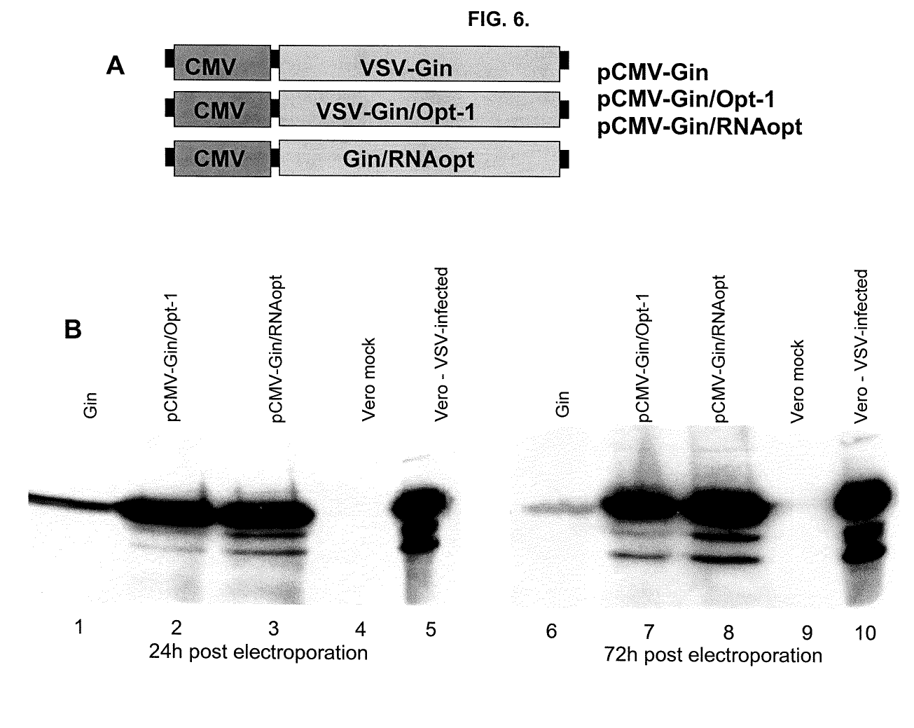

To improve on this finding, two methods of coding sequence optimization were analyzed to determine if they might improve transient expression of VSV G (Indiana serotype; Gin). One method, described as RNA optimization (RNAopt), uses synonymous nucleotide substitutions to increase GC content and disrupt sequence motifs that inhibit nuclear export, decrease translation, or destabilize mRNAs (Schneider, et al. J Virol 71:4892-903, 1997; Schwartz, et al. J Virol 66:7176-82, 1992; Schwartz, et al. J Virol 66:150-9). The second method of optimization is a codon optimization method detailed in Table 1 (Opt-1). The modified coding sequences, as well as the native Gin open reading frame, were then cloned 3′ of the human cytomegalovirus (hCMV) promoter and enhancer from immediate early region 1 (Boshart, et al. Cell 41: 521-30, 1985; Meier and Stinski, Intervirology 39: 331-42, 1996) to produce three vectors (Top FIG. 6A). To compare G protein expression, 50 μg of plasmid DNA was electroporated into approximately 1×10⁷ Vero cells (Witko, et al. J Virol Methods 135:91-101, 2006) and total cellular protein was harvested 24 or 72 hours post-electroporation. Western blot analysis (FIG. 6B) with an anti-VSV polyclonal antiserum revealed that G protein abundance was increased significantly by either optimization method. These results demonstrated that higher and more sustained levels of G protein expression could be achieved in Vero cells by combining electroporation (Witko, et al. J Virol Methods 135:91-101, 2006) with the use of plasmids containing optimized VSV G protein coding sequences.

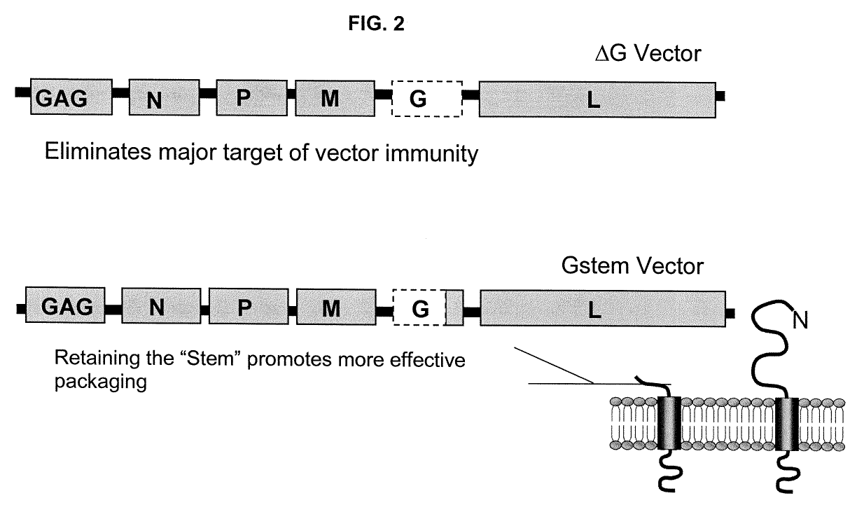

After finding that electroporation of plasmids containing optimized genes produced high levels of G protein expression in Vero cells, studies were conducted to determine whether the increased abundance of G enhanced vector packaging. Important as well in this experiment, a comparison of packaging yields was conducted with ΔG and Gstem vectors (FIG. 2). The Gstem vector was developed because Robison and Whitt (Robison and Whitt, J Virol, 74: 2239-46, 2000) demonstrated that the membrane-proximal extra-cellular 42 amino acids of G protein (the stem region) enhanced particle morphogenesis. Accordingly, it was postulated that a VSV expression vector that expressed a truncated G protein (Gstem) composed of the intracellular domain, the trans-membrane region, and the 42-amino acid extracellular domain might undergo more efficient maturation and improve packaging yields. The results from 4 independent experiments are shown in FIG. 7. Cells were electroporated with plasmid vectors containing the native G sequence (pCMV-Gin, solid or hatched #1 bars), Gin/Opt1 (solid or hatched #2 bars) or Gin/RNAopt (solid or hatched #3 bars), and 24 hours post-electroporation the monolayers were infected with approximately 0.1 IU of rVSV-Gag1-AG (hatched bars) or rVSV-Gag1-Gstem (solid bars). The findings revealed that both plasmids containing optimized sequences promoted more efficient packaging. Yields rose by 0.5 to 1.0 log₁₀ IU for either the ΔG or Gstem vectors as determined by the plaque titration method described by Schnell et al. (Schnell, et al. Cell 90: 849-57, 1997). In addition, the Gstem vector yields were from 0.2 to 1 log₁₀ unit higher than those of ΔG. These results demonstrated that packaging yields as high as 1×10⁸ IUs were attainable when the VSV-Gstem vector was propagated in Vero cells electroporated with plasmid containing an optimized VSV G gene.

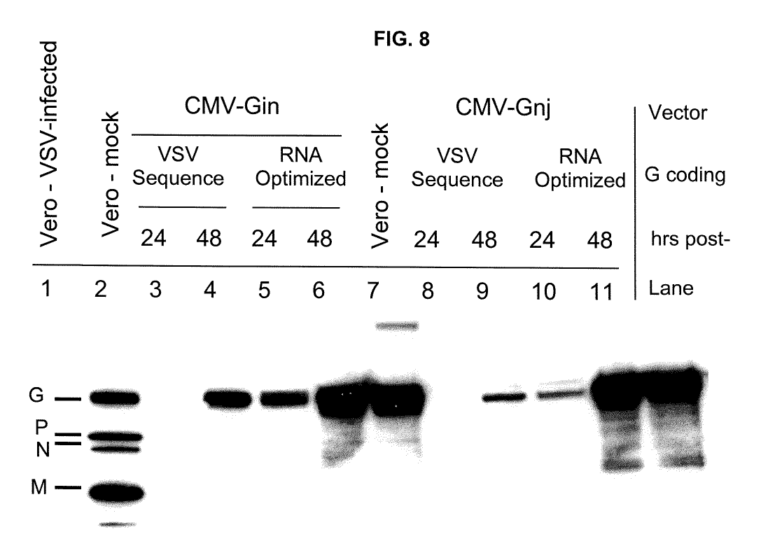

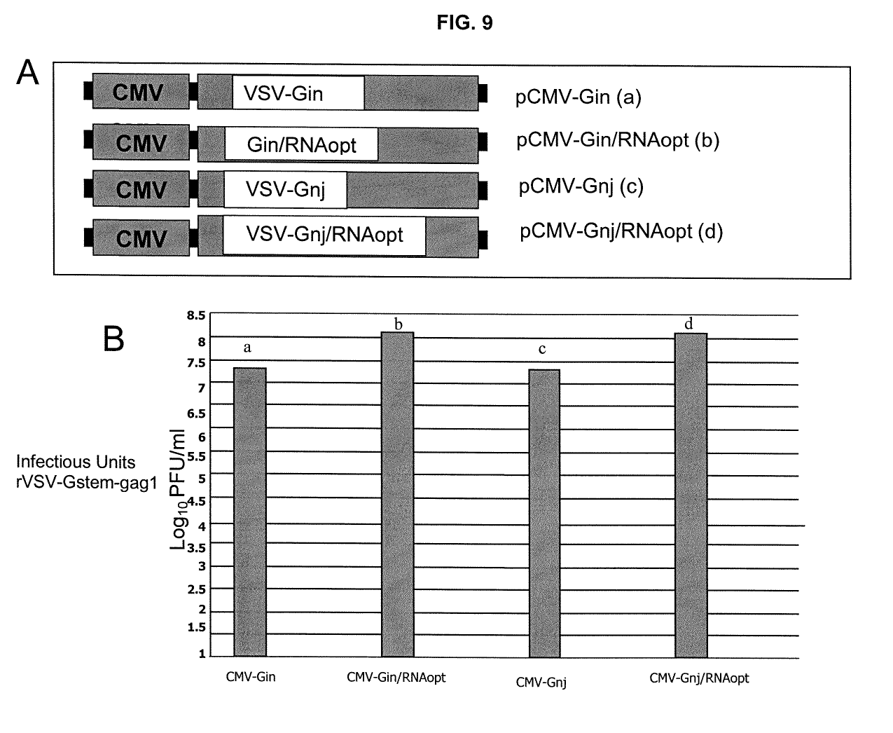

To lessen the effects of anti-vector immunity directed against G protein, live replicating VSV vectors can be produced that encode G proteins derived from different serotypes (Rose, et al. J Virol 74:10903-10, 2000). Similarly, ΔG and Gstem vectors can be packaged with G proteins from different serotypes. To determine if the transient expression packaging method would work readily with a glycoprotein derived from a different strain, plasmid vectors encoding VSV G protein from the New Jersey serotype (Gnj) were constructed with either the native coding sequence or a sequence that was subjected to RNA optimization. The Gnj plasmid vectors were tested first by evaluating transient protein expression after electroporation. FIG. 8 is a Western blot analysis showing a comparison of transient expression of native or optimized VSV G protein coding sequences derived from the New Jersey serotype (Gnj) or Indiana serotype (Gin). Western blot analysis showed that RNA optimization significantly improved the magnitude of Gnj protein expression (FIG. 8) suggesting that pCMV-Gnj/RNAopt would enhance viral vector packaging. When VSV-Gstem-gag1 packaging was tested (FIG. 9), RNA optimization improved yields by about 10-fold boosting particle titers to 1×10⁸ IUs per ml.

### Example

### Rescue of Vesicular Stomatitis Viruses in Vero Cells Via Electroporation-Mediated Transfection

### DNA Preparation:

For each electroporation, the following plasmid DNAs were combined in a microfuge tube: 25-50 μg plasmid expressing T7 (pCl-Neo-Bcl-T7) “hCMV-T7 expression plasmid”, 10 μg VSV Full Length plasmid, 8 μg N plasmid, 4 μg P plasmid, 1 μL plasmid; 1 μM plasmid and 1 μg G plasmid. While working in a biosafety hood, the DNA volume was adjusted to 250 μl with sterile, nuclease-free water. Next, 50 μl of 3M Sodium Acetate (pH 5) was added, and the tube contents were mixed. Subsequently, 750 μl of 100% Ethanol was added and the tube contents were mixed. This was followed by incubation of the tube at −20° C. for 1 hour to overnight. Thereafter, the DNA was pelleted in a microfuge at 14,000 rpm, 4° C. for 20 minutes. While working in a biosafety hood, the supernatant was discarded without disturbing the DNA pellet. Residual ethanol was removed from the tube, and the DNA pellet was then allowed to air dry in a biosafety hood for 5-10 minutes. The dried DNA pellet was resuspended with 50 μl of sterile, nuclease-free water.

### Solutions

The following solutions were employed during cell culture and virus rescue: Trypsin/EDTA, Hank's buffered saline, 1 mg per ml soybean trypsin inhibitor prepared in PBS, and the media shown below in Table 4.

TABLE 4

| Medium 1 | Medium 2 | Medium 3 |
|---|---|---|
| Dulbecco's modified | Iscove's modified Dulbecco's | Dulbecco's modified |
| minimum essential medium | medium (IMDM) | minimum essential medium |
| (DMEM) | | (DMEM) |
| 10% heat-inactivated fetal | 220 μM 2-mercaptoethanol | 10% heat-inactivated fetal |
| bovine serum | (tissue culture grade) | bovine serum |
| 220 μM 2-mercaptoethanol | 1% DMSO (tissue culture | 220 μM 2-mercaptoethanol |
| (tissue culture grade) | grade) | (tissue culture grade) |
| 1% Nonessential amino | 1% Nonessential amino | 1% Nonessential amino |
| acids (10 mM solution) | acids (10 mM solution) | acids (10 mM solution) |
| 1% sodium pyruvate (100 mM | 1% sodium pyruvate (100 mM | 1% sodium pyruvate (100 mM |
| solution) | solution) | solution) |
| | | 50 μg/ml gentamicin |

### Cell Culture and Virus Rescue

Vero cells were maintained in Complete DMEM composed of Dulbecco's Modified Eagle's minimum essential medium (DMEM; Invitrogen or Cellgro) supplemented with 10% heat-inactivated fetal bovine serum (Cellgro), 1% sodium pyruvate (Invitrogen), 1% Nonessential amino acids, and 0.01 mg/ml gentamicin (Invitrogen). This corresponded to Medium 3. Cells were subcultured the day prior to conducting electroporation and incubated at 37° C. in 5% CO₂.

Virus rescue was initiated after introduction of plasmid DNA into Vero cells by electroporation. Optimal conditions for electroporation were determined empirically beginning from conditions recommended for Vero cells by David Pasco in online Protocol 0368 available at www.btxonline.com (BTX Molecular Delivery Systems).

For a single electroporation, Vero cells from a near-confluent monolayer (T150 flask) were washed 1× with approximately 5 ml of Hank's Buffered Saline Solution. Then, the cells were detached from the flask in 4 ml of trypsin-EDTA (0.05% porcine trypsin, 0.02% EDTA; Invitrogen). In particular, after addition of the trypsin/EDTA solution to the monolayer, the flask was rocked to evenly distribute the solution, followed by incubation at room temperature for 3-5 minutes. The trypsin/EDTA solution was then aspirated, and the sides of the flask were tapped to dislodge cells. Medium 1 (10 ml) was then used to collect cells from the flask and the cells were transferred to a 50 ml conical tube. Subsequently, 1 ml of trypsin inhibitor (1 mg/ml) was added to the tube containing the cells and the contents were mixed gently. The cells were collected from the suspension by centrifugation at 300×g for 5 minutes at room temperature after which the supernatant was aspirated and the pellet was resuspended in 10 ml of Medium 2. Next, 1 ml of trypsin inhibitor (1 mg/ml) was added to the cell suspension and the suspension was gently mixed. Subsequently, the cells were collected from the suspension by centrifugation at 300×g for 5 minutes at room temperature. The supernatant was aspirated and the cell pellet was resuspended in a final volume of 0.70 ml of Medium 2.

A 50 μl DNA solution prepared as described above in nuclease-free water, which contained 25-50 μg plasmid expressing T7 (pCl-Neo-Bcl-T7) “hCMV-T7 expression plasmid”, 10 μg VSV Full Length plasmid, 8 μg N plasmid, 4 μg P plasmid, and 1 μg each of L, M and G plasmids, was combined with the 0.7 ml of cell suspension. The cells and DNA were gently mixed and the mixture was transferred to an electroporation cuvette (4 mm gap; VWR or BTX). In some embodiments, the G plasmid contains a non-optimized VSV G coding sequence (e.g., native G open reading frame). In some other embodiments, the G plasmid contains an optimized VSV G coding sequence, such as those described herein. A BTX Square-Wave Electoporator (BTX ECM 820 or 830; BTX Molecular Delivery Systems) was used to pulse the cells (four times, 140-145 V, 70 ms) after which they were incubated at room temperature for approximately 5 min before 1 ml of Medium 1 was added and the cuvette contents were transferred to a sterile 15 ml centrifuge tube containing 10 ml of Medium 1 followed by gentle mixing. Electroporated cells were then collected by centrifugation at 300×g for 5 min at room temperature and resuspended in 10 ml of Medium 1 before transfer to a T150 flask containing 25 ml of Medium 1. The flask was incubated overnight at 37° C., 5% CO₂. The following day, the medium was replaced with 15-30 ml of Medium 3. Incubation was continued at 37° C., 5% CO₂ with periodic medium changes until CPE was evident. VSV replication was typically evident as early as 3-4 days, but in some instances could take as long as 6 days. Also, in some instances, a coculture step was required before cytopathic effect (CPE) was evident.

Coculture was initiated approximately 48-72 h after electroporation by aspirating all but 10 ml of medium from the flask after which the cells were detached by scraping. The detached cells were pipeted multiple times to minimize the sized of the cell aggregates and transferred to a flask containing an established 50%-confluent monolayer of Vero cells that either transiently or constitutively express a VSV G protein encoded by an optimized VSV G gene.

For example, a suitable coculture method employed for rescue of propagation-defective rVSV lacking a functional G protein (ΔG and Gstem viruses) employed a coculture monolayer, which was prepared by first electroporating Vero cells from a confluent T150 flask with 50 μg of a plasmid vector containing an optimized VSV G gene (e.g., pCMV-Opt1 or pCMV-RNAopt from Examples 1 and 2 above). The electroporation was performed as described above. After washing the electroporated cells, the cells were incubated overnight at 37° C., 5% CO₂ to allow for expression of the VSV G protein. The medium was replaced with 15 ml of Medium 3 before establishing the coculture. These cells are referred to herein as “plaque expansion cells”, and are used as a monolayer to establish coculture with the virus rescue cells described in the preceding paragraph.

The monolayer of plaque expansion cells are infected with virus at a multiplicity of infection (MOI) between 0.1 and 0.01 during the coculturing step. The coculture was incubated at 32-37° C., 5% CO₂ until CPE was evident, which generally took about 24 to 48 hours. The virus was thereafter purified by centrifugation through a sucrose cushion using methods well known in the art.

Any articles or references referred to in the specification, including patents and patent applications, are incorporated herein in their entirety for all purposes.

What is claimed is:

1. A method of producing attenuated Vesicular Stomatitis Virus (VSV) in a cell culture, the method comprising:
introducing a plasmid vector comprising an optimized VSV G gene into cells;
expressing VSV G protein from said optimized VSV G gene;
infecting the cells expressing VSV G protein with an attenuated VSV;
growing the infected cells in culture;
recovering the attenuated VSV from the culture.

2. The method of claim 1, wherein the attenuated VSV is a propagation-defective VSV.

3. The method of claim 1, wherein the infecting step comprises coculturing the cells expressing the VSV G protein with cells transfected with: a viral cDNA expression vector comprising a polynucleotide encoding a genome or antigenome of the attenuated VSV; one or more support plasmids encoding an N, P, L and G protein of VSV; and a plasmid encoding a DNA-dependent RNA polymerase.

4. The method of claim 3, wherein the cells are further transfected with a support plasmid encoding an M protein of VSV.

5. The method of claim 3, wherein the cells are transfected via electroporation.

6. The method of claim 3 wherein viral genome-length RNA is transcribed from the polynucleotide encoding the genome or antigenome of the attenuated VSV.

7. The method of claim 3, wherein the DNA-dependent RNA polymerase is T7 RNA polymerase and wherein the viral cDNA expression vector and the support plasmids are under the control of a T7 promoter.

8. The method of claim 3, wherein the VSV G protein encoded by the support plasmid is encoded by a non-optimized VSV G gene.

9. The method of claim 3, wherein the VSV G protein encoded by the support plasmid is encoded by an optimized VSV G gene.

10. The method of claim 1, wherein the expression of VSV G protein from said optimized VSV G gene is under the control of a cytomegalovirus-derived RNA polymerase II promoter.

11. The method of claim 1, wherein the expression of VSV G protein from said optimized VSV G gene is under the control of a transcriptional unit recognized by RNA polymerase II producing a functional mRNA.

12. The method of claim 1, wherein the optimized VSV G gene is derived from an Indiana serotype or New Jersey serotype.

13. The method of claim 1, wherein said optimized VSV G gene is selected from the group consisting of SEQ ID NO: 3, SEQ ID NO: 4 and SEQ ID NO: 5.

14. The method of claim 3, wherein the polynucleotide is operatively linked to a transcription terminator sequence.

15. The method of claim 3, wherein the polynucleotide is operatively linked to a ribozyme sequence.

16. The method of claim 1, wherein the attenuated VSV encodes a heterologous antigen.

17. The method of claim 16, wherein the heterologous antigen is from a pathogen.

18. The method of claim 17, wherein the pathogen is selected from measles virus, subgroup A and subgroup B respiratory syncytial viruses, human parainfluenza viruses, mumps virus, human papilloma viruses of type 1 or type 2, human immunodeficiency viruses, herpes simplex viruses, cytomegalovirus, rabies virus, human metapneumovirus, Epstein Barr virus, filoviruses, bunyaviruses, flaviviruses, alphaviruses, influenza viruses, hepatitis C virus and C. trachomatis.

19. The method of claim 16, wherein the attenuated VSV further encodes a non-viral molecule selected from a cytokine, a T-helper epitope, a restriction site marker, or a protein of a microbial pathogen or parasite capable of eliciting an immune response in a mammalian host.

20. The method of claim 1, wherein the cells are qualified production cells.

21. The method of claim 20, wherein the cells are Vero cells.

22. The method of claim 1, wherein the attenuated VSV lacks a VSV G protein (VSV-ΔG).

23. The method of claim 22, wherein the yield of attenuated VSV is greater than about 1×10⁶ IU per ml of culture.

24. The method of claim 1, wherein the attenuated VSV expresses a G protein having a truncated extracellular domain (VSV-Gstem).

25. The method of claim 24, wherein the yield of attenuated VSV is greater than about 1×10⁶ IU per ml of culture.

26. The method of claim 1, wherein the attenuated VSV expresses a G protein having a truncated cytoplasmic tail (CT) region.

27. The method of claim 26, wherein the attenuated VSV expresses a G protein having a cytoplasmic tail region truncated to one amino acid (G-CT1).

28. The method of claim 26, wherein the attenuated VSV expresses a G protein having a cytoplasmic tail region truncated to nine amino acids (G-CT9).

29. The method of claim 1, wherein the attenuated VSV comprises the N gene which has been translocated downstream from its wild-type position in the viral genome, thereby resulting in a reduction in N protein expression.

30. The method of claim 1, wherein the attenuated VSV contains noncytopathic M gene mutations (Mncp), said mutations reducing the expression of two overlapping in-frame polypeptides that are expressed from the M protein mRNA by initiation of protein synthesis at internal AUGs, affecting IFN induction, affecting nuclear transport, or combinations thereof.

31. A method of producing attenuated Vesicular Stomatitis Virus (VSV) in a cell culture, the method comprising:
transfecting cells with: a viral cDNA expression vector comprising a polynucleotide encoding a genome or antigenome of the attenuated VSV; one or more support plasmids encoding N, P, L and G proteins of VSV; and a plasmid encoding a DNA-dependent RNA polymerase;
growing the transfected cells in culture;
rescuing the attenuated VSV from the culture;
infecting cells expressing VSV G protein encoded by an optimized VSV G gene with the rescued attenuated VSV;
growing the infected cells in culture; and
recovering the attenuated VSV from the culture of infected cells.

32. The method of claim 31, wherein the cells are further transfected with a support plasmid encoding an M protein of VSV.

33. The method of claim 31, wherein the attenuated VSV is a propagation-defective VSV.

34. The method of claim 31, wherein the DNA-dependent RNA polymerase is T7 RNA polymerase and wherein the viral cDNA expression vector and the support plasmids are under the control of a T7 promoter.

35. The method of claim 31, wherein a genome-length RNA is transcribed from the polynucleotide encoding the genome or antigenome of the attenuated VSV.

36. The method of claim 31, wherein the G protein encoded by the support plasmid is encoded by a non-optimized VSV G gene.

37. The method of claim 31, wherein the expression of VSV G protein from said optimized VSV G gene is under the control of a cytomegalovirus-derived RNA polymerase II promoter.

38. The method of claim 31, wherein the expression of VSV G protein from said optimized VSV G gene is under the control of a transcriptional unit recognized by RNA polymerase II producing a functional mRNA.

39. The method of claim 31, wherein the optimized VSV G gene is derived from an Indiana serotype or New Jersey serotype.

40. The method of claim 31, wherein the cells are transfected via electroporation.

41. The method of claim 31, wherein the attenuated VSV encodes a heterologous antigen.

42. The method of claim 31, wherein said optimized VSV G gene is selected from the group consisting of SEQ ID NO: 3, SEQ ID NO:4 and SEQ ID NO: 5.

43. The method of claim 31, wherein the attenuated VSV lacks a VSV G protein (VSV-ΔG).

44. The method of claim 43, wherein the yield of attenuated VSV is greater than about 1×10⁶ IU per ml of culture.

45. The method of claim 31, wherein the attenuated VSV expresses a G protein having a truncated extracellular domain (VSV-Gstem).

46. The method of claim 45, wherein the yield of attenuated VSV is greater than about 1×10⁶ IU per ml of culture.

47. A method of improving the packaging of a propagation-defective Vesicular Stomatitis Virus (VSV) comprising:
a) introducing a plasmid vector encoding an optimized VSV G gene into a cell;
b) transiently expressing VSV G protein from the optimized VSV G gene;
c) introducing a propagation-defective VSV into the cell transiently expressing the VSV G protein;
d) growing cells in culture;
e) recovering the packaged VSV from the culture.

48. An immunogenic composition comprising an immunogenically effective amount of attenuated VSV produced according to the method of claim 1 in a pharmaceutically acceptable carrier.

49. The immunogenic composition of claim 48, wherein the attenuated VSV encodes a heterologous antigen.

50. A composition for producing an attenuated Vesicular Stomatitis Virus (VSV) in a cell culture comprising:
a) a vector that comprises an optimized VSV G gene;
b) a polynucleotide encoding a genome or antigenome of an attenuated VSV; and
c) a vector that encodes a DNA-dependent RNA polymerase.

51. The composition of claim 50, wherein the DNA-dependent RNA polymerase encoded by component c) is a T7 RNA polymerase.

52. The composition of claim 50, further comprising one or more support vectors that encode VSV proteins selected from:
i—an N protein;
ii—a P protein;
iii—an L protein;
iv—an M protein; and
v—a G protein.

53. The composition of claim 50, wherein the attenuated VSV of b) is a propagation-defective VSV.

54. A kit for producing an attenuated Vesicular Stomatitis Virus (VSV) in a cell culture comprising:
a vector that comprises an optimized VSV G gene.

55. The kit of claim 54, further comprising:
a viral cDNA expression vector comprising a polynucleotide encoding a genome or antigenome of an attenuated VSV; and
a vector that encodes a DNA-dependent RNA polymerase.

56. The kit of claim 55, wherein the DNA-dependent RNA polymerase is T7 RNA polymerase.

57. The kit of claim 54, further comprising one or more support vectors that encode VSV proteins selected from:
i—an N protein;
ii—a P protein;
iii—an L protein;
iv—an M protein; and
v—a G protein.
